(19) United States
(12) Patent Application Publication
(10) Pub. No.: US 2017/0020508 A1
(43) Pub. Date: Jan. 26, 2017

(54) Assemblies and Methodologies for Internal Transfascial Mesh Fixation

(71) Applicant: YALE UNIVERSITY, New Haven, CT (US)

(72) Inventor: Kurt E. Roberts, New Haven, CT (US)

(21) Appl. No.: 15/302,521

(22) PCT Filed: May 15, 2014
(86) PCT No.: PCT/US14/38076

§ 371 Date: Oct. 7, 2016

Related U.S. Application Data
(60) Provisional application No. 61/977,683, filed on Apr. 10, 2014.

(51) Int. Cl. A61B 17/04; A61B 17/32

(52) U.S. Cl. CPC A61B 17/0401; A61B 17/32; A61B 2017/0409; A61B 2017/0417; A61B 2017/0464; A61F 2/0063

(57) ABSTRACT

An assembly for securing a mesh patch to a surface of a peritoneum layer of an abdominal wall. The assembly includes first and second holding members coupled by a suture. After the two holding members are deployed from a shaft, tension on the coupler pulls the first holding member and the second holding member towards each other. Various locking arrangements are disclosed to cause the locking of the first and second holding members in position such that a mesh patch that is positioned against the peritoneum layer is secured thereagainst by one of the first and the second holding members while the other of the first and second holding members is retained against the fascia layer of the abdominal wall. Assemblies and methodologies for all of the foregoing are provided.

### BACKGROUND OF THE INVENTION

This invention relates generally to instruments used for repairing hernias and methods for repairing hernias, and in particular, to assemblies and methodologies for internal transfascial fixation of a mesh during a laparoscopic hernia repair procedure.

A hernia is a weakness or tear in the abdominal muscles through which the inner lining of the abdomen pushes the weakened area of the abdominal wall to form a small balloon-like sac. A loop of intestine or abdominal tissue can push into the sac, which can cause a noticeable bulge under the skin. Early on, it may flatten out when the person lies down because it's still a reducible hernia (the hernia contents can go back into the abdomen). Often, the loop of intestine becomes trapped and the person loses the ability to make the bulge flatten out, and a painful nonreducible hernia (abdominal contents are now stuck in the hernia and cannot move back into the abdomen) has formed. The pressure of tissue pushing through the weakened area can cause significant pain and discomfort. The abdominal wall has natural areas of potential weakness that are present from birth. Other areas of weakness develop due to a variety of factors, such as surgery, injury, pregnancy, aging or strain.

There are a number of activities and events that may aggravate a hernia, or may lead to the discovery of a hernia, which include, but are not limited to, lifting, twisting, pulling, muscle strains, weight gain, prostate problems, chronic constipation, or a chronic cough.

There are also several types of hernias. For example:

Groin (inguinal or femoral) hernias occur in the area of the groin. These hernias may cause pain that radiates down the upper thigh or scrotum. Most groin (inguinal) hernias that occur in adults result from strain on abdominal muscles that have been weakened by age or congenital factors. Femoral hernias occur a little bit lower in the groin and are treated the same as inguinal hernias.

Umbilical (belly-button) hernias occur near the navel, which has a natural weakness from the blood vessels of the umbilical cord. These hernias may occur in infants at or just after birth, and may resolve at 3 or 4 years of age. However, the area of weakness can persist throughout life, and can occur in men, women and children at any time. In adults, umbilical hernias will not resolve, and may progressively worsen over time. They are sometimes caused by abdominal pressure due to being overweight, excessive coughing, or pregnancy.

Incisional (ventral) hernias occur near the site of previous trauma to the abdomen or an incision from a prior surgery. These hernias can appear at the site of a previous surgery weeks, months, or even years later, and can vary in size from small to very large and complex. Muscles that heal after an incision can sometimes break down and a hernia can form. A ventral hernia may develop without a previous abdominal incision. Unattended, they may widen and become extremely difficult to repair.

Surgical treatment options for the repair of umbilical ventral or incisional hernia repairs include:

Open hernia repair or the traditional “open” repair, which can be quite difficult and complicated operations. The weakened tissue of the abdominal wall is re-incised and a repair is reinforced most commonly using a prosthetic mesh. It can also be done using a primary repair, i.e. a suture repair without the use of mesh. Complications frequently occur because of the large size of the incision required to perform this surgery. These are primarily wound complications such as a wound infection or an infection of the mesh. Unfortunately, a mesh infection after this type of hernia repair most frequently requires a complete removal of the mesh and ultimately results in surgical failure. In addition, large incisions required for open repair are commonly associated with significant postoperative pain.

Laparoscopic hernia repair is a new method which was developed over the last 20 years. The operation is performed using a surgical telescope and specialized instruments. The surgical mesh is placed into the abdomen underneath the abdominal muscles through small incisions to the side of the hernia. In this manner, the weakened tissue of the original hernia is never re-incised to perform the repair and one can minimize the potential for wound complications such as infections. In addition, performance of the operation through smaller incisions can make the operation less painful and recovery quicker. Laparoscopic repair has been demonstrated to be safe and a more resilient repair than the open hernia repair.

The technique most surgeons perform includes two (2) different but complementing ways of attaching the mesh to the abdominal wall:

Trans-fascial fixation with a suture, wherein the mesh is usually circumferentially fixated to the strength layer of the abdominal wall (i.e. the fascia) at a distance of about 5 to 8 cm circumferentially at the edge of the mesh. This is accomplished by making a skin incision, bringing the suture with a suture passer through the abdominal wall and through the mesh into the abdominal cavity. The suture is then taken out of the suture passer and kept intraabdominal while the suture passer is pulled back close to the skin and then repassed through the abdominal wall at a distance of approximately 1 cm from the previous passing through the fascia and the mesh until it is intraabdominal again. The suture is then placed into the suture passer and brought back out through the abdominal wall. The 2 strings of this suture are then tied so that the mesh is pulled close to the abdominal wall (i.e. the peritoneum). The knot is tied above the fascia and below the skin. The skin is then closed with either a stitch or skin glue.

Tacking the mesh to the peritoneum, wherein the mesh is circumferentially tacked to the peritoneum at a distance of 1 to 2 cm of each other between the transfascial sutures. This is accomplished with a laparoscopic tacking device. The tacks have approximately a 5 mm depth into the peritoneum but do not reach into the fascia.

However, it is perceived that there are deficiencies in the foregoing techniques. Accordingly, improved assemblies for repairing hernias and improved methodologies therefor, that overcomes the drawbacks found in the prior art while also achieving the advantages and objectives as set forth herein, is desired.

### SUMMARY AND OBJECTIVES OF THE PRESENT INVENTION

Generally speaking, preferred embodiments of the present invention are directed to assemblies for securing a mesh patch to a surface of a peritoneum layer of an abdominal wall is provided.

In accordance with a first embodiment, the assembly comprises an outer elongated shaft having a first end; a first holding member at least partially positionable in the outer elongated shaft and a second holding member at least partially positionable in the outer elongated shaft, a coupler for coupling the first holding member to the second holding member, the coupler comprising a locking arrangement and an intermediate portion; a deployment member for deploying at least the first holding member out the first end of the outer elongated shaft, wherein the second holding member is also exitable out the first end of the outer elongated shaft; wherein the locking arrangement is lockable about the intermediate portion of the coupler, and a tension on the intermediate portion of the coupler pulls the first holding member and the second holding member towards each other; whereby with the locking arrangement locked to the intermediate portion of the coupler, a mesh patch that is positioned against the peritoneum layer is secured thereagainst by one of the first and the second holding members while the other of the first and second holding members is retained against the fascia layer of the abdominal wall.

In a specific embodiment of this first embodiment, the first holding member is deployed into a region of the abdominal wall intermediate a skin layer and a fascia layer and the mesh patch is secured against the peritoneum layer by the second holding member while the first holding member is retained against the fascia layer of the abdominal wall.

In accordance with a second preferred embodiment, the assembly comprises an outer elongated shaft having a first end; a first holding member at least partially positionable in the outer elongated shaft and a second holding member at least partially positionable in the outer elongated shaft, a coupling arrangement for coupling the first holding member to the second holding member, the coupling arrangement comprising a first coupler that comprises a first end that is coupled to the first holding member and an intermediate portion that passes through the second holding member; and a second coupler that comprises an intermediate portion and a locking arrangement that is lockable to the intermediate portion of the first coupler; a deployment member for deploying at least the first holding member out the first end of the outer elongated shaft, wherein the second holding member is also exitable out the first end of the outer elongated shaft, wherein a tension on the intermediate portion of the coupler pulls the first holding member and the second holding member towards each other; and locking the locking arrangement on the intermediate portion of the first coupler locks the first holding member in tension relative to the second holding member; whereby a mesh patch that is positioned against the peritoneum layer is secured thereagainst by one of the first and the second holding members while the other of the first and second holding members is retained against the fascia layer of the abdominal wall.

In a particular embodiment of this second embodiment, the first holding member is deployed into a region of the abdominal wall intermediate a skin layer and a fascia layer and the mesh patch is secured against the peritoneum layer by the second holding member while the first holding member is retained against the fascia layer of the abdominal wall.

In accordance with a third preferred embodiment, the assembly comprises an outer elongated shaft having a first end; a first holding member at least partially positionable in the outer elongated shaft and a second holding member at least partially positionable in the outer elongated shaft, a coupling arrangement for coupling the first holding member to the second holding member, the coupling arrangement comprising a female section coupled to one of the first and second holding members, and a male section coupled to the other of the first and second holding members, wherein the male and female sections have complementary locking structures for interlocking engagement therebetween; and a coupler that includes a first end that is coupled to the first holding member and an intermediate portion that passes through the female section, male section and the second holding member; a deployment member for deploying at least the first holding member out the first end of the outer elongated shaft, wherein the second holding member is also exitable out the first end of the outer elongated shaft, wherein the insertion of the male section into the female section causes the first holding member and the second holding member to be interlocked with each other; whereby a mesh patch that is positioned against the peritoneum layer is secured thereagainst by one of the first and the second holding members while the other of the first and second holding members is retained against the fascia layer of the abdominal wall.

In a specific embodiment of this first embodiment, the mesh patch is secured against the peritoneum layer by the second holding member while the first holding member is retained against the fascia layer of the abdominal wall.

The present invention is also directed to methodologies for securing a mesh patch to a surface of a peritoneum layer of an abdominal wall utilizing assemblies as disclosed herein.

For example, using the assembly as disclosed in the first embodiment, the method comprises the steps of inserting the first end of the outer elongated shaft through the mesh layer and through at least the peritoneum layer and the fascia layer of the abdominal wall; deploying the first holding member out the first end of the outer elongated shaft and into a region of the abdominal wall intermediate a skin layer and a fascia layer; pulling the outer elongated shaft out of the abdominal wall; exiting the second holding member out the first end of the outer elongated shaft; applying tension on the intermediate portion of the coupler to pull the first holding member and the second holding member towards each other; and locking the locking arrangement about the intermediate portion of the coupler, whereby a mesh patch that is positioned against the peritoneum layer is secured thereagainst by the second holding member while the first holding member is retained against the fascia layer of the abdominal wall.

Another preferred methodology uses the assembly as disclosed in the second embodiment, and comprises the steps of inserting the first end of the outer elongated shaft through the mesh layer and through at least the peritoneum layer and the fascia layer of the abdominal wall; deploying the first holding member out the first end of the outer elongated shaft and into a region of the abdominal wall intermediate a skin layer and a fascia layer; pulling the outer elongated shaft out of the abdominal wall; exiting the second holding member out the first end of the outer elongated shaft; applying a tension on the intermediate portion of the first coupler to pull the first holding member and the second holding member towards each other; and locking the locking arrangement about the intermediate portion of the first coupler; whereby a mesh patch that is positioned against the peritoneum layer is secured thereagainst by the second holding member while the first holding member is retained against the fascia layer of the abdominal wall.

Yet another preferred methodology uses the assembly as disclosed in the third embodiment, and comprises the steps of inserting the first end of the outer elongated shaft through the mesh layer and through at least the peritoneum layer and the fascia layer of the abdominal wall; deploying the first holding member out the first end of the outer elongated shaft and into a region of the abdominal wall intermediate a skin layer and a fascia layer; pulling the outer elongated shaft out of the abdominal wall; exiting the second holding member out the first end of the outer elongated shaft; inserting the male section into the female section so that the first holding member and the second holding member are in interlocked engagement with each other; whereby a mesh patch that is positioned against the peritoneum layer is secured thereagainst by the second holding member while the first holding member is retained against the fascia layer of the abdominal wall.

Still further, in accordance with yet additional embodiments of the presentation invention, the presently disclosed assemblies could be used in connection with respective alternative methodologies in which, generally speaking, the first end of the outer elongated shaft is inserted through the fascia layer of the abdominal wall and thereafter through at least the peritoneum layer and the mesh layer, with specifics of such methodologies being disclosed more fully herein

Accordingly, it is an object of the present invention to achieve a more precise, exact and secure placement of the mesh to the abdominal wall.

It is also an object of the present invention to reduce wound infections and subsequent mesh infections after a procedure to repair a hernia.

Still further it is an object of the present invention to reduce the number of needed incisions on the abdominal wall during a hernia repair.

Still further it is an object of the present invention to provide a more exact placement of the transfascial fixation at the edge of the mesh.

Still further it is an object of the present invention to provide a more effective, more reliable, efficient, secure and safer attachment of the mesh to the fascia in patients having a thick subcutaneous wall.

Still further it is an object of the present invention to reduce the risk of a wound infection and subsequent mesh infection by virtue of the elimination of the sutures being placed through the skin.

Still further it is an object of the present invention to save hospital procedure or operation time because of the ease of use of the present invention and performance of the methodologies disclosed herein.

Moreover, another object of the present invention is to provide for an increase in patient satisfaction for all the reasons noted herein.

Yet another object of the present invention is to provide methodologies and assemblies for carrying out the methodologies that yields fewer complications like bleeding and chronic pain than achievable with prior art assemblies and methodologies.

Still further, another object of the present invention is to facilitate the ability of basic laparoscopic surgeons to perform a more complex advanced laparoscopic surgery than heretofore possible.

Still other objects and advantages of the invention will in part be obvious and will in part be apparent from the specification.

The invention accordingly comprises the features of construction, combination of elements, sequence of steps and arrangement of parts which will be exemplified in the construction and methodology hereinafter set forth, and the scope of the invention will be indicated in the claims.

### BRIEF DESCRIPTION OF THE DRAWINGS

For a fuller understanding of the invention, reference is had to the following description taken in connection with the accompanying drawings, in which:

FIG. 1 is a perspective view in part, cross-sectional view in part and plan view in part of an assembly constructed in accordance with a first embodiment of the present invention;

FIG. 2 is a view of the assembly of FIG. 1 having been inserted through layers of the abdominal wall and prior to deployment of the first holding member, in accordance with the present invention;

FIG. 3 illustrates the assembly of FIG. 1 as the first holding member is being deployed;

FIG. 4 illustrates the assembly of FIG. 1 having been retracted so as be put in position for the exiting of the second holding member out of the outer shaft;

FIG. 5 illustrates the assembly of FIG. 1 after the second holding member has exited the outer shaft;

FIG. 6 illustrates the first and second holding members of the first embodiment in position such that the mesh patch is retained against the peritoneum layer by the second holding member with the first holding member being retained against the fascia layer of the abdominal wall;

FIG. 7 is a perspective view in part, cross-sectional view in part and plan view in part of an assembly constructed in accordance with a second embodiment of the present invention;

FIG. 8 is a view of the assembly of FIG. 7 having been inserted through layers of the abdominal wall and prior to deployment of the first holding member, in accordance with the present invention;

FIG. 9 illustrates the assembly of FIG. 7 as the first holding member is being deployed;

FIG. 10 illustrates the assembly of FIG. 7 after the second holding member has exited the outer shaft;

FIG. 11 illustrates the first and second holding members of the second embodiment in position such that the mesh patch is retained against the peritoneum layer by the second holding member with the first holding member being retained against the fascia layer of the abdominal wall;

FIG. 12 is a perspective view in part, cross-sectional view in part and plan view in part of an assembly constructed in accordance with yet a third embodiment of the present invention;

FIG. 13 illustrates the assembly of FIG. 12 having been inserted through layers of the abdominal wall and prior to deployment of the first holding member, in accordance with the present invention;

FIG. 14 illustrates the assembly of FIG. 12 as the first holding member is being deployed;

FIG. 15 illustrates the assembly of FIG. 12 having been retracted so as be put in position for the exiting of the second holding member out of the shaft;

FIG. 16 illustrates the assembly of FIG. 12 after the second holding member has exited the outer shaft;

FIG. 17 illustrates the first and second holding members of the third embodiment in position such that the mesh patch is retained against the peritoneum layer by the second holding member with the first holding member being retained against the fascia layer of the abdominal wall; and

FIG. 18 is a view of the first and second holding members retaining a mesh patch against the peritoneum layer, but which has been accomplished by one or more yet further alternative methods in which the various assemblies of the present invention have been inserted in the direction opposite to that illustrated above, namely, that the respective assemblies are first inserted through the fascia layer of the abdominal wall and thereafter through at least the peritoneum layer and the mesh layer. Therefore, generally speaking, in these alternative embodiments, the mesh patch is secured against the peritoneum layer by the first holding member while the second holding member is retained against the fascia layer of the abdominal wall. While only FIG. 18 is provided, and which most closely resembles using the assembly as disclosed in FIG. 1, those skilled in the art would most readily understand that the assemblies illustrated in FIGS. 7 and 12 could be used similarly, although the final completed attachment method, because of the differing coupling of the holding members, would be slightly different. However, FIG. 18 should be recognized as generically disclosing all the alternative methods herein.

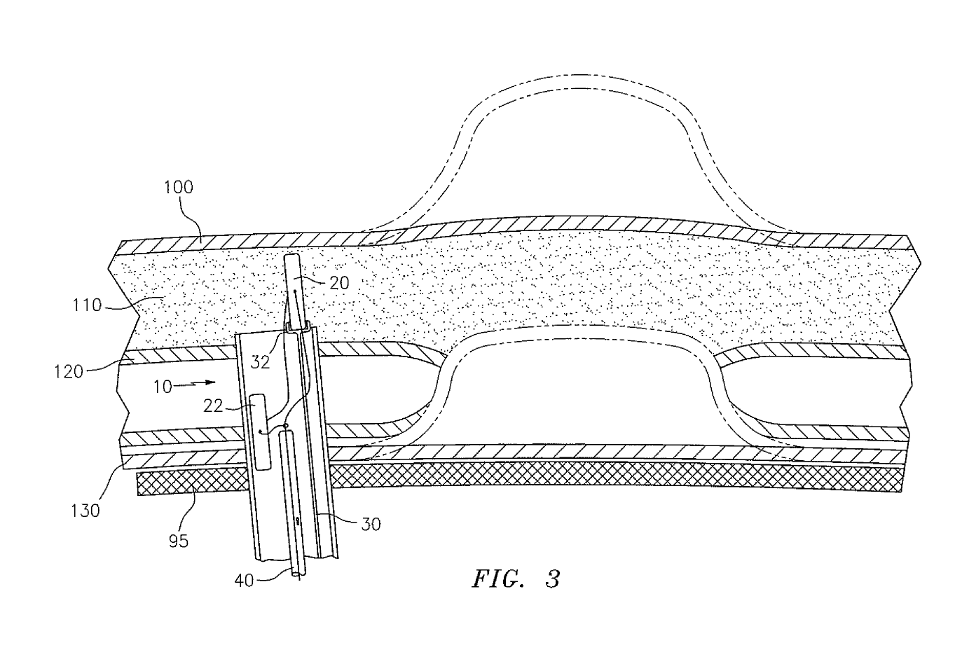

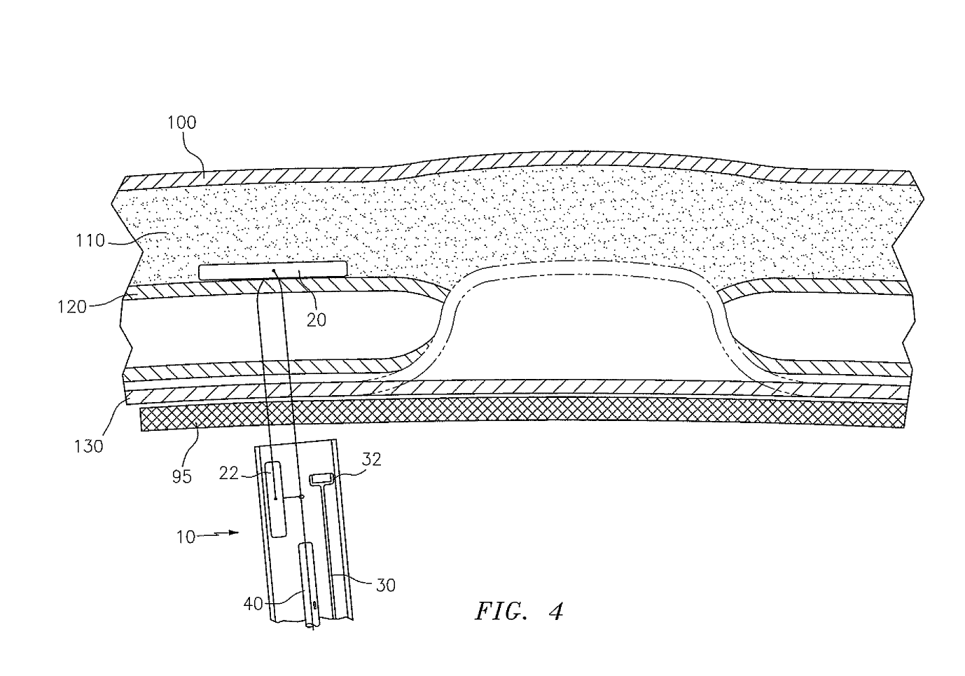

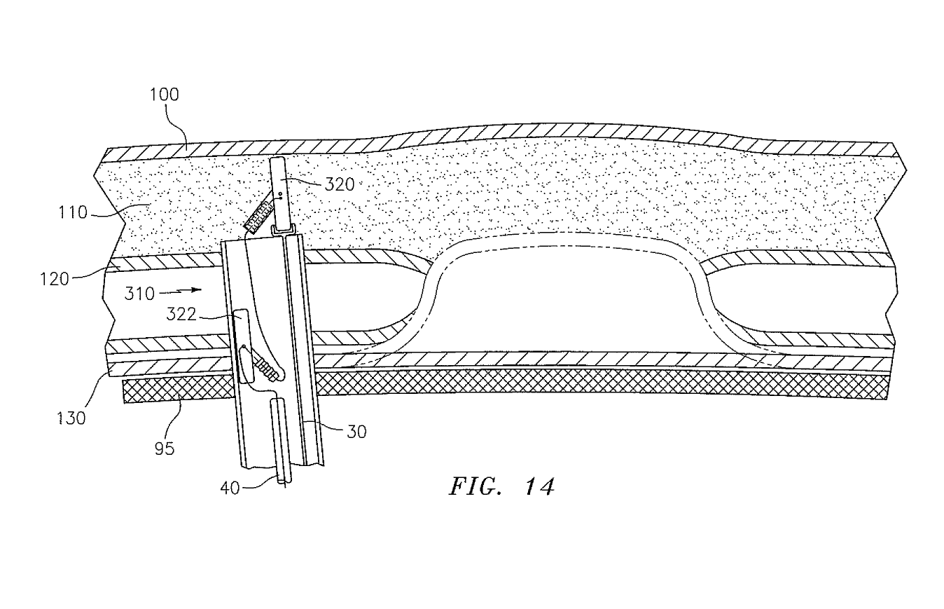

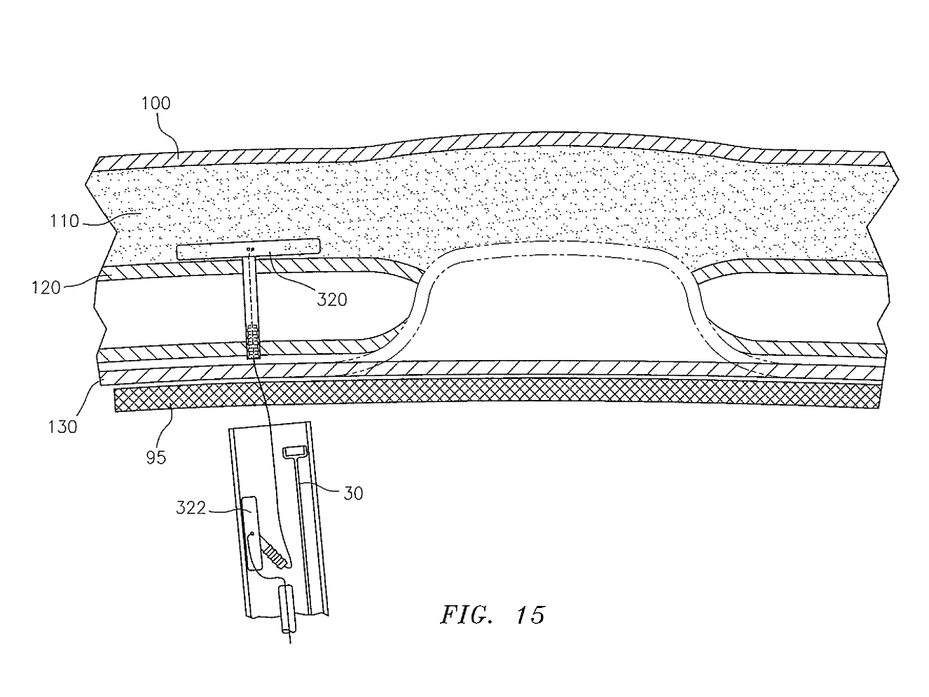

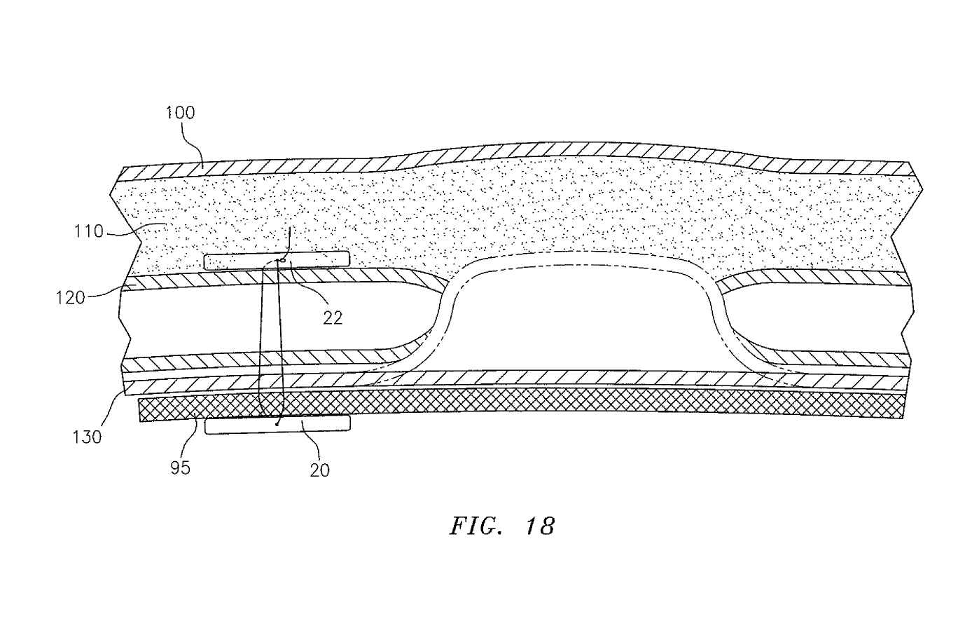

In addition, it will be recognized after a review of the current specification, that the figures are not all in scale among themselves or therebetween. For example, and not limitation, it can be seen that holding member 20 changes in size/scale between that which is illustrated in FIG. 3 and FIG. 4. In a similar manner, holding member 320 changes in size/scale between that which is illustrated in FIG. 14 and FIG. 15. Moreover, if each feature was illustrated to scale within each figure, there would be a need for larger paper or smaller images. Therefore, for the convenience of the reader, the scale has been adjusted for ease of understanding. Specific dimensions for components are provided where appropriate and/or helpful. Where not provided, it is readily assumed that those skilled in the art will understand the features and relative sizes disclosed herein. Lastly, like numbers to identify like parts and features will be used among the various figures, but not all features will be specifically identified in each illustration.

### DETAILED DESCRIPTION OF THE PREFERRED EMBODIMENTS

Generally speaking, it is known that patients frequently complain of the multitude of skin incisions they have after a laparoscopic ventral hernia repair. Most commonly, there are somewhere between 7 to 11 small laparoscopic or small stab incisions that are needed for the procedure. To avoid the multitude of these incisions, preferred embodiments of the present invention utilize a reduced number of incisions to reduce the risk of infections. Additionally, a single port ventral hernia repair technique that permits the use only one (1) small incision is feasible. The preferred embodiments of the present invention provide a transfascial mesh fixation through laparoscopic ports without unneeded/additional skin incisions. The assemblies in accordance with the preferred embodiments and the methodologies using such assemblies provide for the penetration of the mesh, then the peritoneum, then the muscle and the external fascia. Within the subcutaneous tissue before penetration of the skin, the first of the two hereinafter described holding rods is deployed. This may be accomplished through one or two movable deployment members that allow the first holding member to be moved from a vertical position to a horizontal position. Preferably, by pulling on the suture that is attached to the first holding member, the holding member gets pulled closer to the external fascia and lodges just above the external fascia. The intraabdominal/second holding member is then released and then horizontally pushed/placed to the mesh. Hereinafter, preferred embodiments to carry out the foregoing and other aspects of the invention will be disclosed. For example, disclosed hereinafter will be differing suture fixations of the two (2) holding members to keep the mesh in place with the tie either lying above or below the mesh. Pulling down of the upper holding member directly with a suture or pulling of the lower holding member via a pulley system with the fulcrum at the upper/first holding member advantageously allows the approximation of the holding members and subsequent mesh fixation. Additionally the pushing of the lower holding member can allow the suture to be tightened more easily and with the appropriate tension so that a safe and secure attachment of both holding members and the mesh occurs. Alternatively a self-locking zip-tie mechanism can be used as more fully disclosed herein.

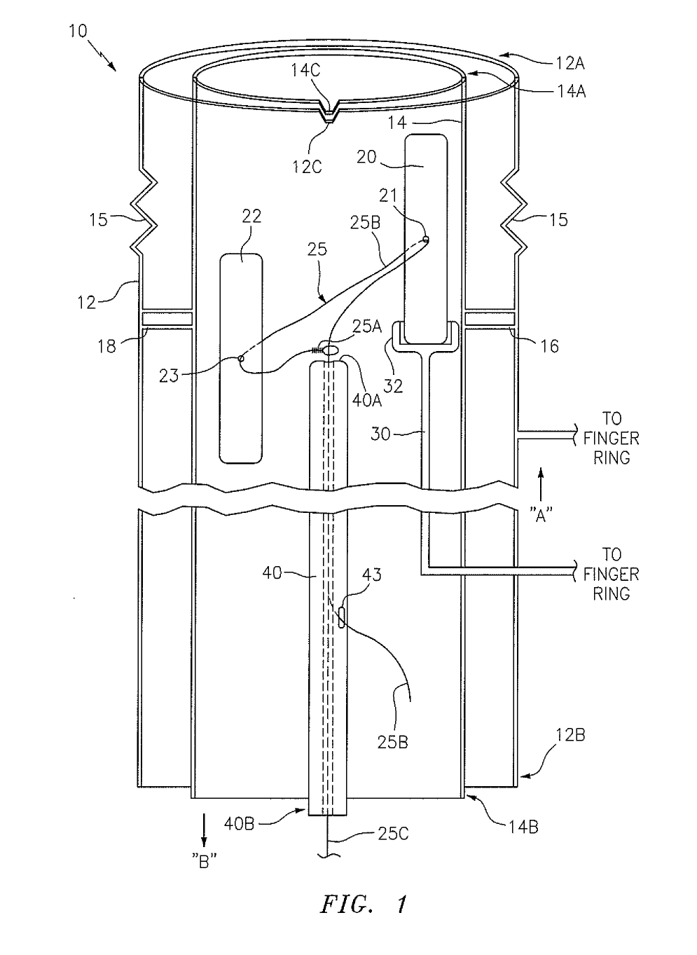

In view thereof, reference is first made to FIG. 1, which illustrates an assembly, generally indicated at 10, for securing a mesh patch to a surface of a peritoneum layer of an abdominal wall, constructed in accordance with a first embodiment of the present invention. In accordance with this first embodiment, assembly 10 preferably comprises a hollow tube section, generally indicated at 12. Positioned inside hollow tube section 12 is an outer elongated shaft, generally indicated at 14, which, in this first embodiment is preferably connected to tube section 12 by connectors, generally indicated at 16, 18. Connectors 16, 18 may be as simple as one or more connecting rods that are glued, coupled, integral with and/or otherwise adhered or connected between the outer surface of outer elongated shaft 14 and the inner surface of tube section 12. Connectors assist in maintaining the connection between tube section 12 and outer elongated shaft 14. Tube section 12 is preferably made of stainless steel, with a diameter, for example at a front end 12A, of approximately 5 mm.

As would be understood in the art, either the circular edge of front end 12A of tube section 12 or the front end 14A of the shaft 14 may be used as the cutting edge for cutting through any soft tissue and the abdominal wall as contemplated herein. Therefore, a spring 15 may extend about the circumference of the wall of the tube IA section 12 as appropriate to ensure the proper pressure is applied in the various embodiments, and just as importantly, to reduce the risk of unintentional and/or undesirable cutting of internal organs or which may otherwise occur during the procedure being described herein. In a preferred embodiment, it is end 14A that is the preferred cutting edge. The construction and/or selection of a suitable spring member or assembly would be understood by those skilled in the art. In fact, the construction set forth in this paragraph is known in the art as a Veress needle.

Outer elongated shaft 14 includes a front end and a rear end, generally indicated at 14A and 14B, respectively. Outer elongated shaft 14 is preferably made of stainless steel at the front end 14A and the remainder being in plastic, with a diameter at front end 14A of approximately 2-3 mm.

In this first embodiment, assembly 10 also comprises a first holding member 20, which is at least partially positionable in the elongated shaft 14. Preferably, first holding member 20 is positionable completely in shaft 14, but it is contemplated that portion of first holding member 20 may extend outward from front end 14A of shaft 14 as insertion into the abdominal wall is proceeding. For example, in such an embodiment, it is the first holding member 20 that extends partially out of the front end 14A and itself has a cutting edge for piercing through the peritoneum and fascia layers of the abdominal wall. This optional configuration is applicable for all first holding members disclosed herein.

Assembly 10 also comprises a second holding member 22, which similarly, is at least partially (and most preferably, completely) positionable in the outer elongated shaft 14. In the embodiment of FIG. 1, and prior to their urging or deployment out of shaft 14 as will be discussed below, both the first and second holding members 20, 22 are completely within shaft 14. The particular relative positioning of the first and second holding members 20, 22 in FIG. 1 is exemplary, as those skilled in the art could position holding members 20, 22 within the shaft 14 differently from that illustrated herein while still remaining within the scope of the present invention.

As also illustrated in FIG. 1, a coupler, generally indicated at 25, couples the first holding member 20 to the second holding member 22. In a preferred embodiment, coupler 25 is a suture as would be understood in the art, but it should be understood that other forms of couplers could be used, including, but not limited to, thread, string, wire, cable, or any other material that can carry out the functionality thereof as explained herein. As used herein, the term “coupler” may be a one-piece assembly (i.e. a suture) or may be comprised of various components that are otherwise coupled or connected to each other as would be understood in the art. In this preferred embodiment, coupler 25 includes a locking arrangement 25A and an intermediate portion 25B. Locking arrangement 25A may be a fastener, clip or other locking device such as a slip-knot. In the preferred embodiment, locking arrangement 25A is a self-tightening knot 25A, and reference thereto will be made hereinafter. For the ease of description, the entire length of coupler 25, other than the portion that comprises knot 25A will be deemed and construed as the intermediate portion 25B for ease of description for purposes of the description and claims.

It should be pointed out that there are several contemplated embodiments of the coupler 25 for coupling the first and second holding members 20, 22. For example, in the embodiment of FIG. 1, self-tightening knot 25A extends out of a through-hole 23 in second holding member 22, with the intermediate portion 25B of coupler 25 extending through the rear of through-hole 23 and then through a through-hole 21 in first holding member 20 as illustrated in FIG. 1. In this way, and as discussed below, a pulling on the intermediate portion 25B, for example, from an end region of intermediate portion 25B, generally indicated at 25C, will cause the knot 25A to self-tighten and lock upon intermediate portion 25B. Through-hole 23 is sized to prevent knot 25A from receding therethrough. To be sure, there is no particular specific region that must be designated “end region” 25C. Rather, it is only for convenience that it should be understood that pulling on any section of coupler 25 extended out of through-hole 21 will be understood to be the “intermediate portion” and carry out the function of tightening the knot 25A as discussed herein. This concept should be understood in to be applicable in all embodiments.

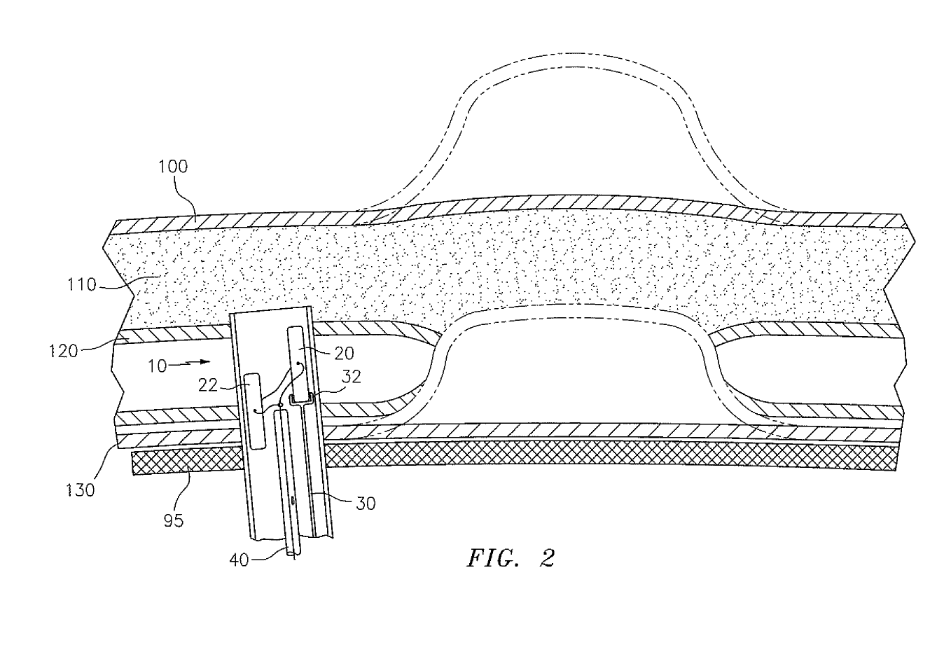

Assembly 10 also comprises a deployment member, generally indicated at 30, for deploying at least the first holding member 20 out the front end 14A of the outer elongated shaft 14 and into a region of the abdominal wall intermediate the skin layer and a fascia layer. FIG. 2 illustrates an exemplary position of assembly 10 after it has passed through the mesh patch, generally indicated at 95, and after it has pierced through the peritoneum later 130 and all layers of the abdominal wall up through the fascia (i.e. “strength”) layer 120, but prior to the first holding member 20 being deployed out of shaft 14.

In a preferred embodiment, deployment member 30 is a pusher that may comprise a first end 32 having a “cup” or “U” shape so as to engage and deploy an end of first holding member 20 out of the front end 14A of shaft 14. FIG. 3 illustrates first holding member 20 having been deployed out of shaft 14 sufficient for it to be freely movable in the region intermediate the fascia later 120 and the skin layer 100. For example and as illustrated in FIG. 1, a sufficient elongated channel may be formed in tube section 12 and shaft 14 to permit deployment member 30 to move in the direction indicated by arrow A and thus deploy first holding member 20 out of the shaft 14.

If the holding member 20 does not quickly and independently lie flush against the fascia layer as illustrated in FIG. 4, a simple jostling or toggling of the coupler may be needed to ensure that first holding member 20 lies flush against the fascia layer as illustrated in FIG. 4.

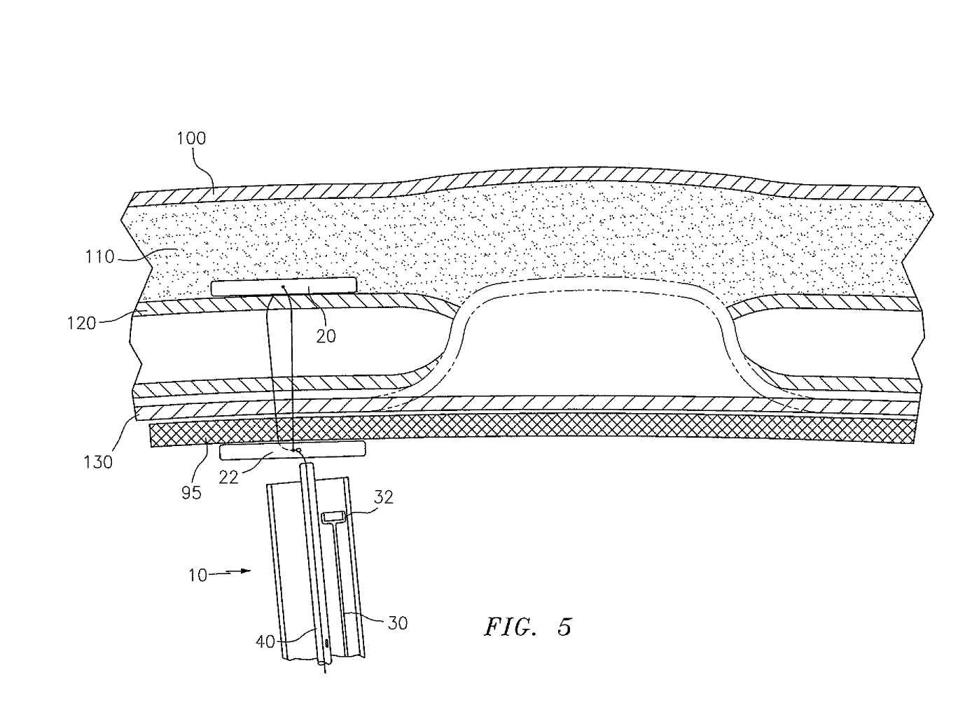

Next, assembly 10 and outer elongated shaft 14 will be withdrawn out of the abdominal wall to the position generally indicated in FIG. 4, where it will now be desirable to have second holding member 22 also exited out the front end 14A of the outer elongated shaft 14. The exiting of second holding member 22 out the distal end 14A of the outer elongated shaft 14 can be achieved in a variety of ways. For example, second holding member 22 can be pushed out the distal end 14A of the shaft 14 (as discussed below) or it can be pulled passively by the knot 25A of coupler 25. Using either of these two preferred ways or any other way consistent with the present invention, second holding member 22 will thus now lie within the inner cavity and below the mesh later as illustrated in FIG. 5. To be sure, deployment member 30 can be but need not be used to deploy second holding member 22 and this applies to the deployment of all second holding members disclosed herein. To be sure, deployment member 30 is depicted as a pusher designed to be integrated into assembly 10. However, it is contemplated that deployment member 30 in all embodiments may similarly be a pusher insertable from a rear end of shaft 14, which can likewise achieve its functionality as disclosed herein.

In particular FIG. 5 illustrates second holding member 22 after just having been exited from shaft 14 (dotted lines). To position second holding member 22 against the mesh layer 95, an inner elongated shaft 40 may be provided in shaft 14. Inner elongated shaft 40 is positionable by sliding into shaft 14 via the rear end 14B. In addition, at least a section of intermediate portion 25B of the coupler 25 extends within inner shaft 40 as illustrated in FIG. 1. The front end 40A of shaft 40 has an opening sufficient to allow intermediate portion 25B to be pulled therethrough yet small enough to preferably prevent knot 25A from falling therein. In this way, the tip of inner elongated shaft 40 can facilitate in maintaining the position of the knot 25A proximate the second holding member 22 and permits the tightening of the self-tightening knot 25A by pushing on knot 25A as the intermediate portion 25B of the coupler 25 is pulled out and from the rear end of the inner elongated shaft 40. Alternatively (or in addition), inner elongated shaft 40 may include an opening or slot 43 in a side surface out of which the intermediate portion 25B of the coupler 25 is pullable (see FIG. 1) so as to apply tension on the intermediate portion of the coupler so as to cause the first holding member to be pulled towards the second holding member, as discussed below. FIG. 1 shows both alternatives together, but obviously, portion 25B will either extend out the rear end or side surface of shaft 40, but cannot do so simultaneously.

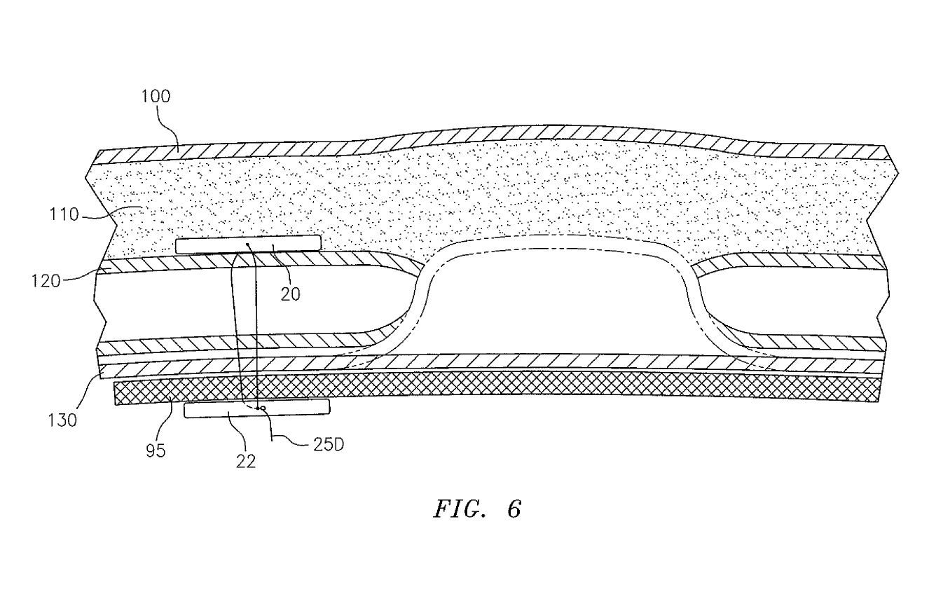

FIG. 6 illustrates the arrangement of the holding members 20, 22 against the fascia layer 120 and the mesh layer 95, respectively, after the coupler 25 has been cut at position 25D. As should now be understood, it is also possible to design assembly 10 such that second holding member 22 may also be pushed out of shaft 14 by a slight manipulation and/or angling of either the deployment member 30 and/or shaft 40.

Specifically, a tension on the intermediate portion 25B of the coupler 25 (e.g. by pulling on a section of the intermediate portion 25B that extends out of the rear 40B end or opening 43 of shaft 40) causes (i) the first holding member 20 and the second holding member 22 to be pulled towards each other and (ii) the tightening of the self-tightening knot 25A about the intermediate portion 25B of the coupler 25. In this way, the mesh patch 95 that is positioned against the peritoneum layer 130 is secured thereagainst by the second holding member 22 being in locked engagement with the first holding member 20, which is itself retained against the fascia layer 120 of the abdominal wall.

As can thus be seen, the locking arrangement 25A is lockable about the intermediate portion 25B of the coupler 25, and a tension on the intermediate portion of the coupler causes the first holding member 20 to be pulled towards the second holding member 22, such that with the locking arrangement locked to the intermediate portion 25B of the coupler 25A, a mesh patch 95 that is positioned against the peritoneum layer 130 is secured thereagainst by the second holding member 22 while the first holding member 20 is retained against the fascia layer 130 of the abdominal wall. Those skilled in the art would understand how to utilize a clip or other fastener in place of the preferred knot assembly.

As can thus also be seen, the preferred embodiments above comprise means for engaging the locking arrangement 25A while tension is applied to the intermediate portion 25B of the coupler 25. In a preferred embodiment, the means for engaging comprises the inner elongated shaft 40 that is positionable in the outer elongated shaft and through which at least a section of the intermediate portion of the coupler extends. In this way, the inner elongated shaft facilitates in maintaining the position of the locking arrangement 25A proximate the second holding member 22 as tension is applied to the intermediate portion 25B of the coupler 25. Alternatively, with the tubular section 12, the means for engaging may comprise a slot 12C (see FIG. 1) in a front end 12A of the tubular section 12, wherein the slot 12C maintains the position of the locking arrangement 25A proximate the second holding member 22 as tension is applied to the intermediate portion 25B of the coupler 25. To be sure however, while not optimal, the means for engaging may also comprise a slot 14C in a front end of the outer elongated shaft, wherein the slot 14C would likewise maintain the position of the locking arrangement proximate the second holding member as tension is applied to the intermediate portion of the coupler.

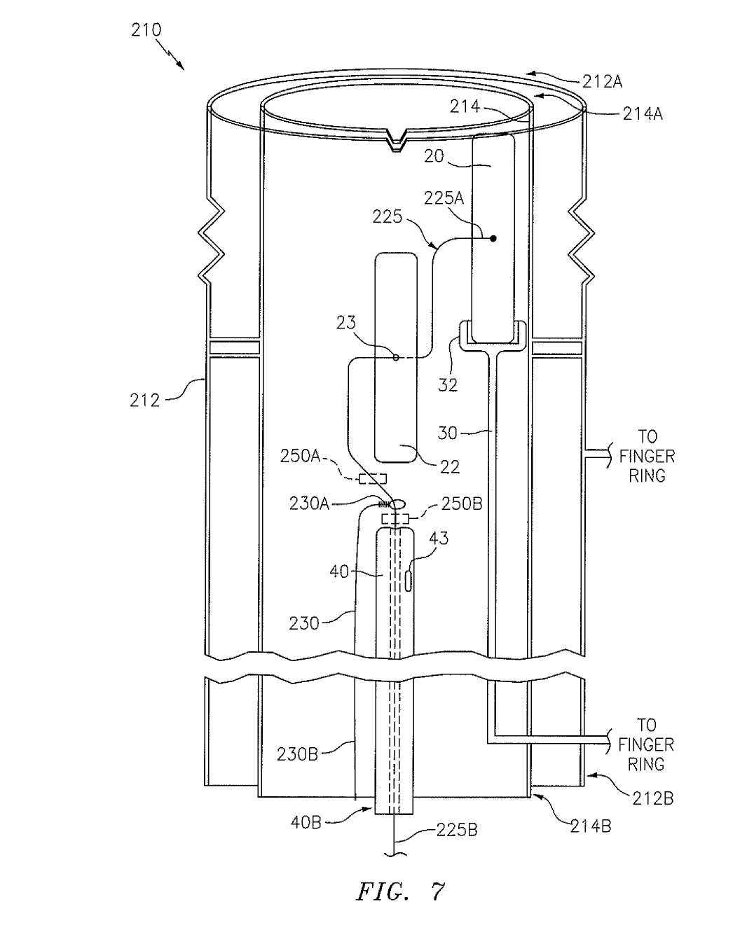

Reference is now made to FIG. 7, which illustrates an assembly, generally indicated at 210, for securing a mesh patch to a surface of a peritoneum layer of an abdominal wall, constructed in accordance with a second embodiment of the present invention. In accordance with this second embodiment, assembly 210 preferably comprises a hollow tube section, generally indicated at 212. Likewise, positioned inside hollow tube section 212 is an outer elongated shaft, generally indicated at 214, which is also preferably connected to tube section 212 by connectors, as in the first embodiment. Preferably, tube section 212 and shaft 214 are identically constructed as tube section 12 and outer elongated shaft 14, respectively.

In this second embodiment, assembly 210 likewise comprises a first holding member 20 and a second holding member 22 both positioned as in the first embodiment.

Differing in this second embodiment for example, is the coupling arrangement for coupling the first holding member 20 to the second holding member 22. In this embodiment, the coupling arrangement may comprise a coupler 225 that may likewise be a suture as would be understood in the art, but it should be understood that other forms of couplers could be used, including, but not limited to, thread, string, wire, cable, or any other material that can carry out the functionality thereof as explained herein. However, in this preferred embodiment, the coupling arrangement also comprises a second coupler, generally indicated at 230, which has a locking arrangement 230A at one end thereof, which similarly may be a clip, slip knot or fastener or the like. A preferred embodiment provides that locking arrangement 230A is also a self-tightening knot 230A, and reference will be made thereto. In this embodiment, a first end 225A of coupler 225 is locked, connected and/or otherwise coupled to first holding member 20, in ways within the scope of the skilled artisan. Coupler 225 then passes through a through-hole 23 in second holding member 22 and preferably extends into and through, as in the first embodiment, an inner elongated shaft 40 and out a rear end thereof, as disclosed above. Alternatively (or in addition), inner elongated shaft 40 may include an opening 43 in a side surface out of which the intermediate portion 225B of the coupler 25 is pullable (see FIG. 1) so as to apply tension on the intermediate portion of the coupler so as to cause the first holding member to be pulled towards the second holding member, as discussed below.

In this preferred embodiment, coupler 225 passes though the locking arrangement (i.e. self-tightening knot) 230A of second coupler 230 as illustrated in FIG. 7. However, as should be understood, if locking arrangement 230A is not a knot but rather a clip or other fastener, then coupler 225 may not need to pass through the knot as should now be understood in the art.

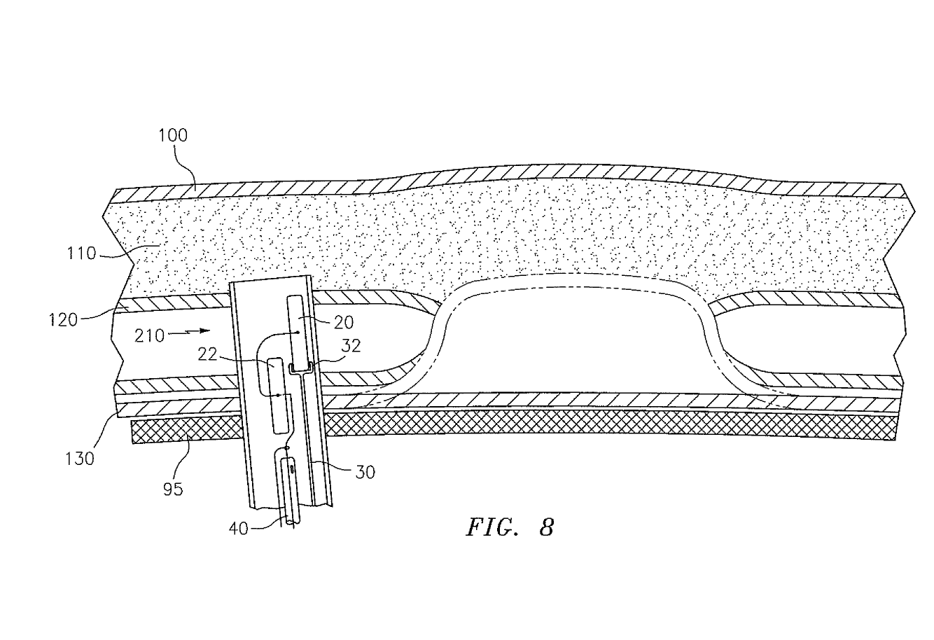

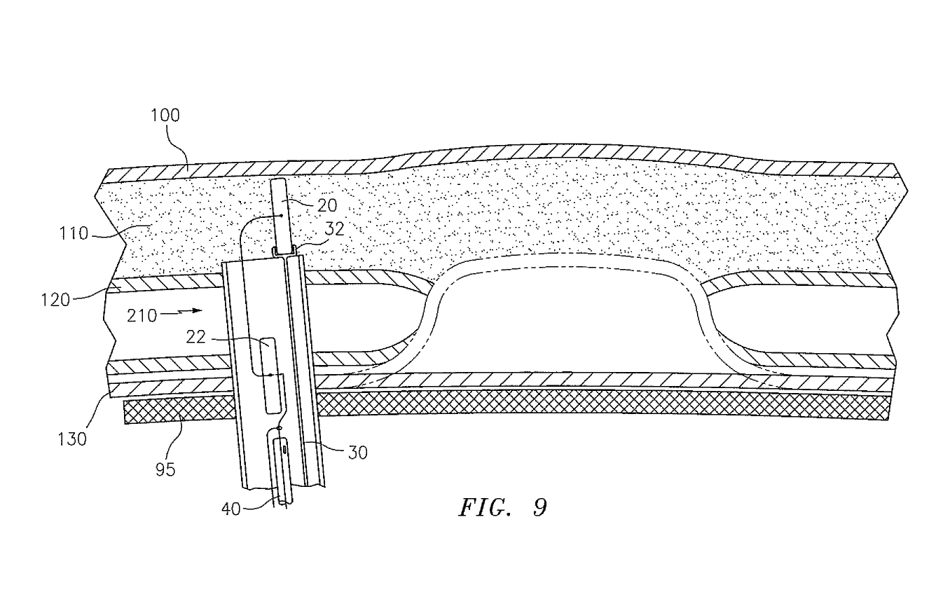

Assembly 210 also comprises a deployment member, generally indicated at 30, for deploying at least the first holding member 20 out the front end of the outer elongated shaft 214 and into a region of the abdominal wall intermediate a skin layer and a fascia layer in the same manner as the urging assembly 30 of the first embodiment. In this way, FIG. 8 illustrates an exemplary position of assembly 210 after it has passed through the mesh patch and after it has pierced through the peritoneum later 130 and all layers of the abdominal wall up through the fascia (i.e. “strength”) layer 120, but prior to the first holding member 20 being deployed out of shaft 214, similar to that of FIG. 2. And similar to FIG. 3, FIG. 9 likewise illustrates first holding member 20 having been deployed out of shaft 214 sufficient for it to be freely movable in the region intermediate the fascia later 120 and the skin layer 100. A sufficient elongated channel may likewise also be formed in tube section 212 and shaft 214 to permit urging assembly 30 to move in the direction indicated by arrow “A” (see FIG. 7) and thus urge first holding member 20 out of the shaft 214.

Similarly, if first holding member 20 does not automatically lie flush against the fascia layer as illustrated in FIG. 4, a simple jostling or toggling of the coupler 225 is all that should be needed to ensure first holding member 20 to lie flush against the fascia layer as needed.

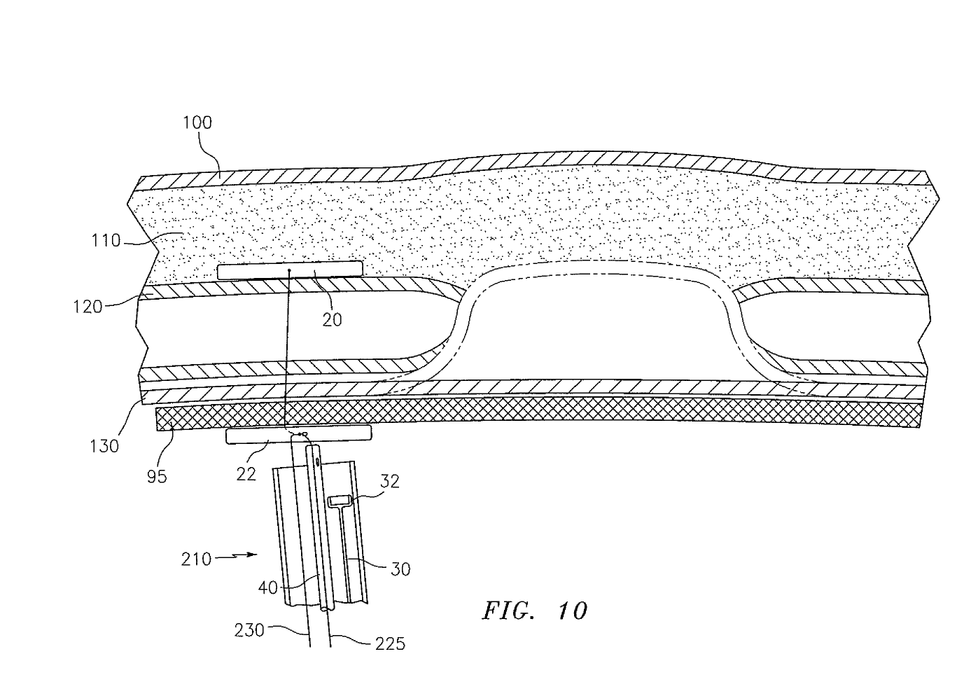

Similarly, after assembly 210 has been withdrawn out of the abdominal wall to the position generally illustrated earlier in FIG. 4, second holding member 22 also is exited out the front end 214A of the outer elongated shaft 214, most preferably by being urged out by the tip of inner shaft 40 or it can simply “fall out” as would be understood by those skilled in the art. In any of the preferred, disclosed or other possible ways, second holding member 22 will thus now lie within the inner cavity and below the mesh layer as illustrated in FIG. 10.

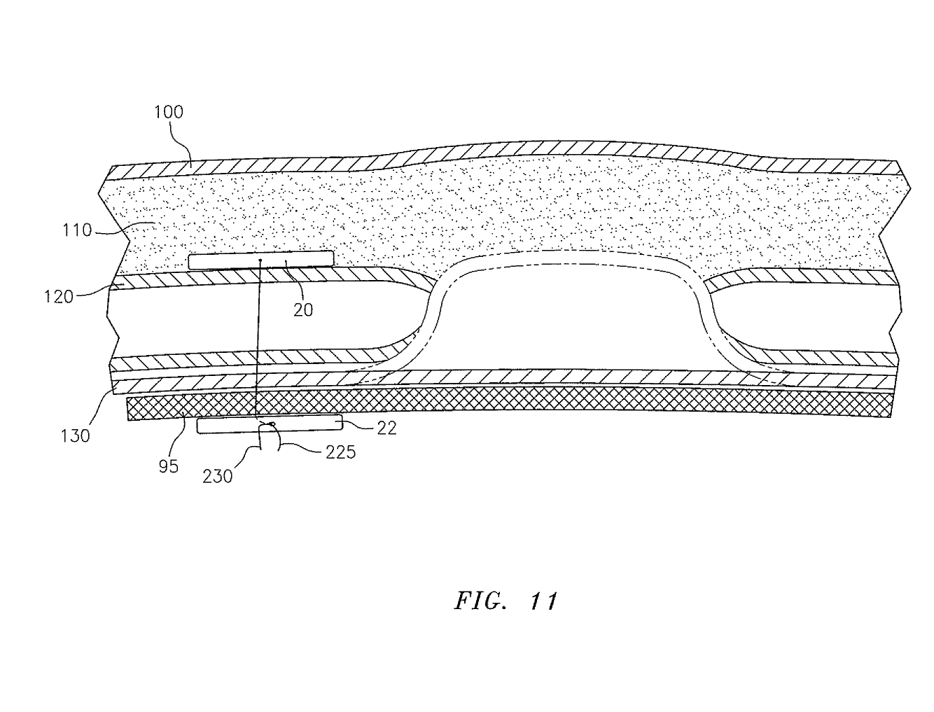

In particular FIG. 10 also illustrates second holding member 22 after just having been released from shaft 214 (dotted lines). To tighten second holding member 22 against the mesh layer 95, an inner elongated shaft 40 may be provided in outer shaft 214B. Similarly, inner elongated shaft 40 is positionable by sliding into shaft 214 via the rear end 214B. The intermediate portion 225B of the coupler 225 may extend within inner shaft 40 as illustrated in FIG. 7 (i.e. and out a rear end or side surface thereof). Likewise, the front end 40A of shaft 40 has an opening sufficient to allow intermediate portion 225B to be pulled therethrough yet small enough to preferably prevent knot 230A of second coupler 230 from falling therein. In this way, if used, the tip 40A of inner elongated shaft 40 facilitates in maintaining the position of the knot 230A of the second coupler 230 proximate the second holding member 22 and permits the tightening of the self-tightening knot 230A by pushing on knot 230A as the intermediate portion 225B of the coupler 225 and the intermediate portion 230B of second coupler 230 are both pulled from the rear end 214B of shaft 214. FIG. 11 similarly illustrates the arrangement of the holding members 20, 22 against the fascia layer 120 and the mesh layer 95, respectively, after the first and second couplers 225, 230 have been cut. Similarly, it is also possible to design assembly 210 such that second holding member 22 may also be pushed out of shaft 214 by a slight manipulation of the urging member 30.

Again, it is noted that the preferred embodiments above comprise means for engaging the locking arrangement 230A while tension is applied to the intermediate portion 225B of the coupler 225. Similar to the first mentioned embodiment, here the means for engaging may likewise comprise the inner elongated shaft 40 that is positionable in the outer elongated shaft and through which at least a section of the intermediate portion of the coupler extends. That is, the inner elongated shaft facilitates in maintaining the position of the locking arrangement 230A proximate the second holding member 22 as tension is applied to the intermediate portion 225B of the coupler 225. Alternatively, with the tubular section 12, the means for engaging may comprise a slot 12C in a front end 12A of the tubular section 12 and/or the means for engaging may comprise a slot 14C in a front end of the outer elongated shaft, all as discussed above.

Here too, it can be seen that a tension on the intermediate portion of the first coupler 225 causes the first holding member 20 and the second holding member 22 to be pulled towards each other and a tension on the intermediate portion 230B of the second coupler 230 causes the tightening of the self-tightening knot 230A about the intermediate portion 225B of the first coupler 225. In this way, the mesh patch 95 that is positioned against the peritoneum layer 130 is secured thereagainst by the second holding member 22 being in locked engagement with the first holding member 20, which is retained against the fascia layer 120 of the abdominal wall.

An optional stopper 250A or 250B (FIG. 7) may also be slidingly provided along coupler 225 and positioned either above or below the knot 230A of second coupler 230 to further assist in locking the first and second holding members 20, 22 in place as set forth herein. Positioned below the knot as with stopper 250B, the means for engaging can push thereagainst to urge knot 230A up towards second holding member 22 prior to pulling on the intermediate portion 225B of coupler 230 so as to tighten the knot 230A on intermediate portion 225B of coupler 225.

It can thus be seen that a tension on the intermediate portion of the first coupler causes the first holding member to be pulled towards the second holding member; and locking the locking arrangement on the intermediate portion of the first coupler locks the first holding member in tension relative to the second holding member, such that a mesh patch that is positioned against the peritoneum layer is secured thereagainst by the second holding member while the first holding member is retained against the fascia layer of the abdominal wall.

It can also be seen that with a self-tightening knot, the intermediate portion 225B of the first coupler 225 passes through the locking arrangement 230A of the second coupler 230, such that the tension on the intermediate portion of the second coupler causes the tightening of the self-tightening knot about the intermediate portion of the first coupler. The cutting of the end(s) of the couplers 225, 230 as shown in FIG. 11 as a final step once the holding members are in position is applicable to this embodiment.

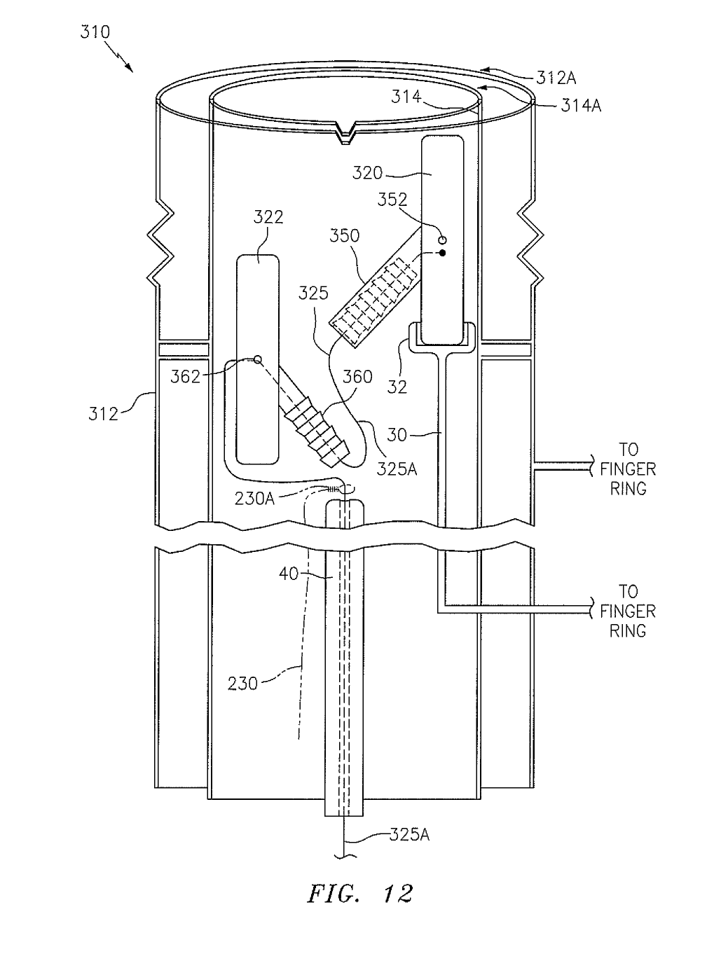

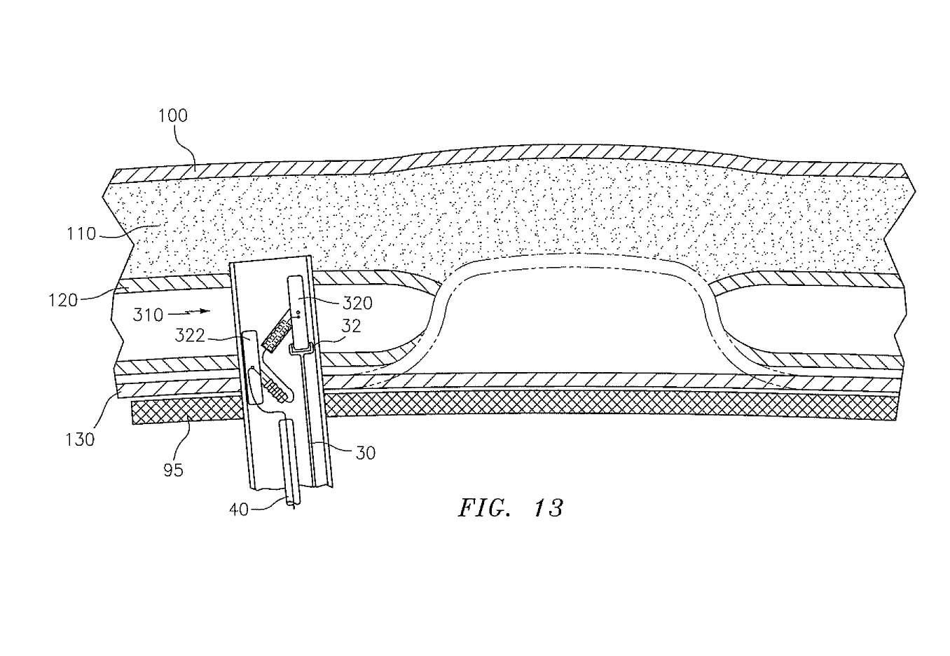

Reference is now made to FIG. 12 (and the sequence of steps illustrated in FIGS. 13-17), wherein another assembly, generally indicated at 310, for securing a mesh patch to a surface of a peritoneum layer of an abdominal wall, is provided. In this alternative embodiment, the assembly 310 likewise preferably comprises a hollow tubular section 312 and an outer elongated shaft 314, both of which are also constructed in identical manners to their respective counterparts of the aforementioned embodiments.

Similarly, this alternative embodiment provides for a first holding member 320 at least partially positionable in the outer elongated shaft 314 and a second holding member 322 at least partially positionable in the outer elongated shaft 314. While the preferred embodiment of FIG. 12 provides that both the first and second holding members are completely disposed within outer elongated shaft 314 prior to being deployed out, it is likewise possible that first holding member 320 is partially exposed with the cutting edge itself.

In this embodiment, there likewise is a coupling arrangement for coupling the first holding member 320 to the second holding member 322. Here, the coupling arrangement comprises a female section, generally indicated at 350, coupled to one of the first and second holding members, and a male section, generally indicated at 360, coupled to the other of the first and second holding members, wherein the female section 350 and the male section 360 have complementary locking structures for interlocking engagement therebetween. In other words, either the female section 350 is coupled to the first holding member 320 and the male section 360 is coupled to the second holding member 322 or the female section 350 is coupled to the second holding member 322 and the male section 360 is coupled to the first holding member 320. In the embodiment illustrated in FIG. 12, the female section 350 is coupled to the first holding member 320 and the male section 360 is coupled to the second holding member 322. As for the preferred complementary locking structures for interlocking engagement therebetween, a zip-tie structure or some other “evergreen tree structure” is preferred, although variations thereon would be understood by those skilled in the art and are thus contemplated herein.

The coupling arrangement also comprises a coupler 325 that includes a first end that is coupled to the first holding member 320 and an intermediate portion 325A that passes through the female section 350, the male section 360 and the second holding member 322, although not necessarily in that order as it depends on the construction, as contemplated above. Therefore, no particular order is meant to be implied and therefore should not be implied herein or in the claims.

The assembly 310 of this alternative embodiment would also preferably provide that the respective female section 350 and male section 360 include hinge structures, indicated by reference numbers 352 and 362 respectively. Such hinge structures would be beneficial to allow both the holding member(s) and its respective female and/or male sections to fit within shaft 314 during the shaft's insertion into the abdominal wall. The hinge structures would also have to be sufficiently resilient, to prevent over rotation and/or over flexing as the holding members were deployed as disclosed herein. For example, the hinge structures may include a locking assembly to lock the respective members 350, 360 in their respective orthogonal positions relative to the holding member to which they are associated. It should also be noted that the position of the respective members 350, 360 in FIG. 12 is exemplary, and such members may be rotated differently while remaining within the scope of the invention.

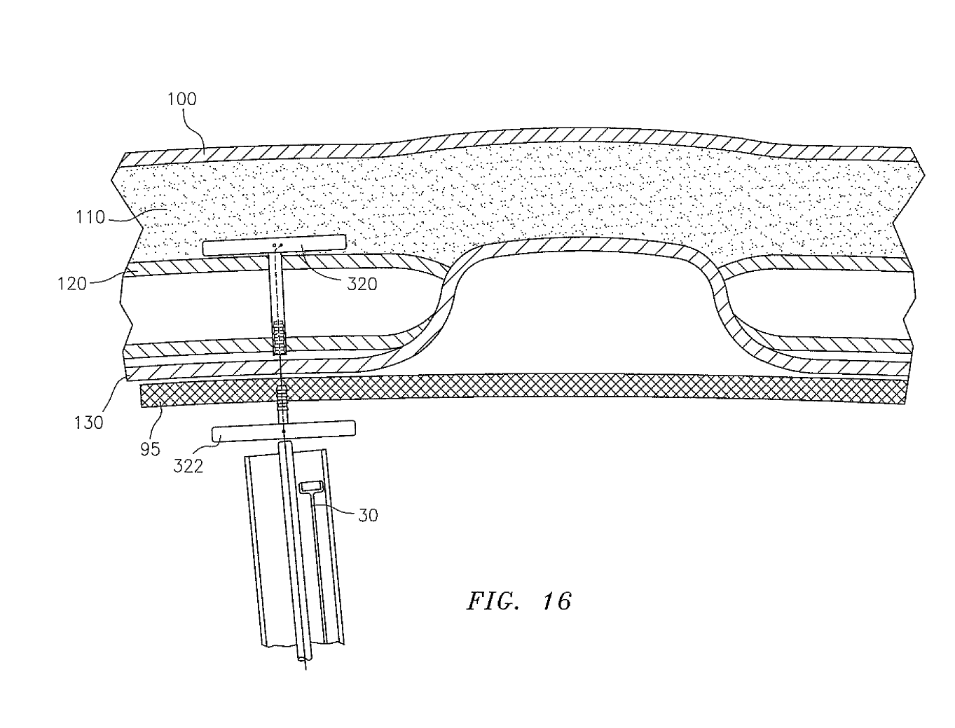

Similar to the earlier embodiments, a deployment member 30 preferably deploys at least the first holding member 320 (see FIGS. 13, 14) out the first end of the outer elongated shaft 314 and into a region of the abdominal wall intermediate a skin layer and a fascia layer, wherein the second holding member 322 is also exitable (see FIGS. 15, 16) out the first end 314A of the outer elongated shaft 314 in a manner similar to all those manners disclosed above with reference to the first two embodiments. Here the deployment of the first holding member 320 (FIGS. 13, 14) most likely must be done deeper in the fatty region between the fascia and skin layers so as to ensure that the associated member 350 or 360 can rotate into position. For this reason, hinges 352, 362 may also include a spring mechanism to ensure proper engagement into position.

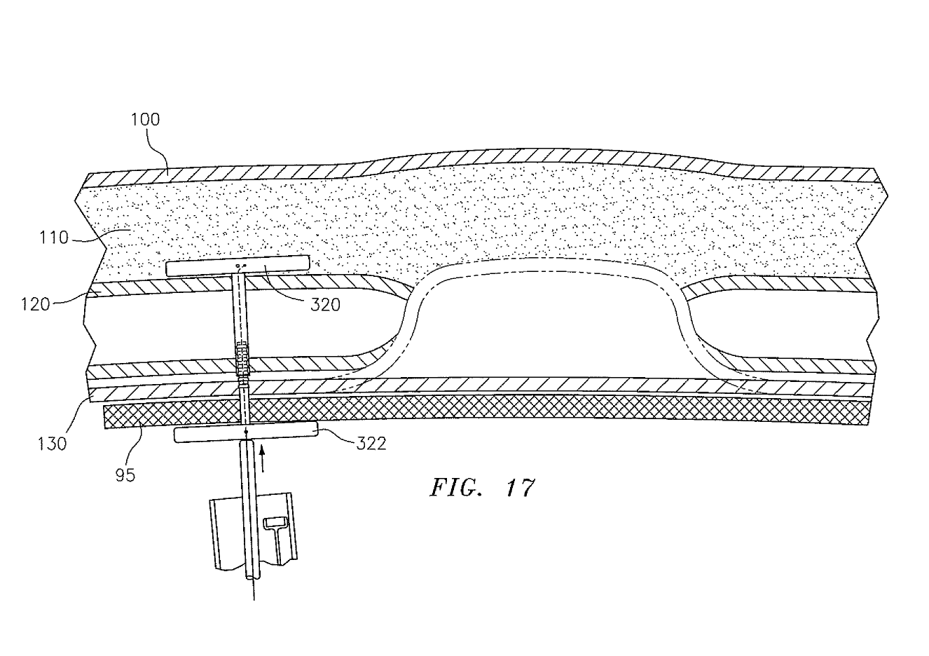

An inner elongated shaft 40 may again be used to and is similarly positionable in the outer elongated shaft 314, which has the intermediate portion 325A of the coupler 325 extending therethrough, and may be used, among other things, to urge the male section 360 to be inserted into the female section 350 by pushing on the second holding member 322 as illustrated in FIG. 17.

In this way, while pulling on the intermediate portion 325A, the urging of the male section 360 into the female section 350 causes the first holding member 320 and the second holding member 322 to be interlocked with each other, whereby a mesh patch that is positioned against the peritoneum layer is retained thereagainst by the second holding member 322 being in engagement with the first holding member 320, which is retained against the fascia layer of the abdominal wall. The cutting of the suture as a final step once the holding members are in position is also applicable to this embodiment.

In this alternative embodiment, a second coupler (e.g. suture) with a locking knot, similar to the preferred embodiment of FIG. 7, may also be used for further assurances of the locking of the first and second holding members together.

Therefore, it can be seen that in this further embodiment, a means for engaging a separate locking arrangement, such as locking arrangement 230A may be provided in this embodiment as well, wherein the means for engaging may comprise all the different structures and features as disclosed above, such as the use of slots in the outer shaft and/or tubular section or using the inner elongated shaft as disclosed above. Similarly, the inner elongated shaft may thus comprise at least one of a rear end out of which the intermediate portion of the coupler is pullable so as to apply tension on the intermediate portion of the coupler so as to cause the first holding member to be pulled towards the second holding member; and an opening in a side surface out of which the intermediate portion of the coupler is pullable so as to apply tension on the intermediate portion of the coupler so as to cause the first holding member to be pulled towards the second holding member.

The means for urging may also comprise a front end of the outer elongated shaft or the front end of the tubular section. Likewise, in a preferred embodiment, the intermediate portion of the coupler 325A may pass through the locking arrangement 230A of the locking coupler 230, and with the locking arrangement preferably being a self-tightening knot, a tension on the intermediate portion of the locking coupler causes the tightening of the self-tightening knot about the intermediate portion of the first coupler.

With the above three (3) main assembly constructions disclosed, many variations and alternative embodiments are contemplated herein.

For example, within any of the outer shafts 14, 214, 314 a separate movable and/or retractable cutting knife may be provided for the initial cutting of the soft tissue and the abdominal wall layers as disclosed herein. Such a cutting knife would need to be able to be at least movable and/or retractable so that the first and second holding members could be deployed as set forth herein. Therefore, applicable to any of the aforementioned embodiments is the available inclusion of a cutting edge for piercing through the peritoneum and fascia layers of the abdominal wall, wherein the cutting edge is movable and/or retractable relative to the front end of the outer elongated shaft after piercing through the abdominal wall of the abdomen for facilitating the deployment of the first and second holding members out of the outer elongated shaft. Alternatively, the first holding member in any of the aforementioned embodiments may include a cutting edge for piercing through the peritoneum and fascia layers of the abdominal wall. In this alternative, it could be the first holding member that would slightly extend out of the outer elongated shaft prior to deployment. It should also be understood that the deployment member 30 can be a simple pusher that is inserted into shaft 14, 314 from the rear.

In other alternative embodiments, the deployment member of any of the aforementioned embodiments may deploy the first holding member out the first end of the outer elongated shaft by pushing from a first end or a side of the first holding member. From the side therefore, it is also contemplated that the deployment member be hinged and may engage a groove on a side or end of the first holding member.

In addition, the deployment member 30 of any of the foregoing embodiments may be shaped so as to engage any of the aforementioned first holding member 20, 320 along a side surface thereof, rather than from an end thereof. For example, the first holding member 20, 320 may include a groove which receives the U-shaped end of the urging member 30. In this way, a spring loaded hinged deployment member 30, as contemplated in this alternative embodiment, for example, could assist in both deploying the first holding member 20, 320 and urging it into its horizontal position for lying against the fascia layer as illustrated above. Likewise, the first holding member could be loaded in the respective shafts 14, 214, 314 in a slightly angled position so as to facilitate the counterclockwise rotation thereof once deployed.

Several alternatives are contemplated with the locking arrangement as well. For example, the locking arrangements of each of the embodiments may be positioned either above or below the second holding member. The self-tightening knot could be one continuous suture of the coupler to which it is attached. Alternatively, other knot configurations are contemplated herein. Further, it is further contemplated that the knot can be sized (e.g. made thicker or thinner) to accommodate the hole in the second holding rod. The hole in the second holding road (e.g. in the first two embodiments) can also be notched so that the knot fits thereon and lies flush with the second holding member surface. Alternatively, the knot can simply lie against the outer surface. Other wedge mechanisms can be used to ensure that the knot does not get pulled through the hole in the second holding member.

To be sure, different features of the different embodiments disclosed herein can be complementary used with each other. That is, the embodiments herein have been generally disclosed, but a variation or alternative shown in one embodiment could be used in any one of the other disclosed embodiments as would be understood by those skilled in the art.

Unless otherwise specified, the materials from which the aforementioned assemblies are constructed would be well understood in the art. For example, the outer tube and outer elongated shafts are preferably made from stainless steel or plastic as would be understood by those skilled in the art. The first and second holding members are preferably of stainless steel or plastic or absorbable or non-absorbable material as would be understood in the art. The deployment member and the inner elongated shaft are also preferably made from plastic as would also be understood in the art.

The foregoing assemblies can thus be used in connection with preferred methodologies in accordance with the present invention. For example, and generally speaking, the assemblies disclosed herein are designed to be able to be placed thru a trocar and secure the mesh from the intraabdominal approach to the fascia. Keeping in mind that some of the features in the figures are not drawn to scale so as to assist the reader, the following is set forth and applicable for all of the embodiments disclosed herein. For example, and generally speaking, the tip of the assembly (approx. 5 to 12 mm in diameter) is placed against the mesh. The assembly (approx. 2 to 8 mm in diameter, length up to 5 cm) is then advanced thru the mesh, peritoneum and fascia. Then the first holding member (length is approx. 0.5 to 3 cm, thickness is approx. 1 to 5 mm) is deployed as disclosed above into the subcutaneous space. The assembly is then pulled back out as set forth above. The second holding member (length is approx. 0.5 to 3 cm, thickness is approx. 1 to 5 mm) is then deployed as disclosed above. The second holding member is coupled to the suture (absorbable or nonabsorbable, might be barbed or not), which in at least the first embodiment disclosed above, includes a locking assembly, and preferably a sliding loop knot (e.g. “Meltzer” knot or other modification thereof). This tie is going through the tissue upwards towards the first holding member and through the first holding member, e.g. through hole 21 of FIG. 1, and then out the assembly for grasping by the surgeon as explained above. The foregoing dimensions should be understood to be applicable to each of the embodiments disclosed herein.

The first holding member acts as a fulcrum. The suture in the abdominal cavity is then pulled, and by pulling thereon, the second holding member is pulled towards the mesh layer.

Once the two holding members are pulled towards each other the sliding loop knot closes and fixates the two holding members in position. As a final step the suture that was pulled intraabdominally is then cut by a scissors or other cutting edge. The cutting of the suture as a final step once the holding members are in position is applicable to all of the embodiments disclosed herein.

In this way, the mesh layer is securely attached towards the abdominal wall through the first and second holding members keeping the fascia and the mesh securely in place.

More particularly, using the assembly as disclosed in the first embodiment, a first preferred method comprises the steps of inserting the first end of the outer elongated shaft through the mesh layer and through at least the peritoneum layer and the fascia layer of the abdominal wall; deploying the first holding member out the first end of the outer elongated shaft and into a region of the abdominal wall intermediate a skin layer and a fascia layer; pulling the outer elongated shaft out of the abdominal wall; exiting the second holding member out the first end of the outer elongated shaft; applying tension on the intermediate portion of the coupler to pull the first holding member and the second holding member towards each other, and locking the locking arrangement about the intermediate portion of the coupler, whereby a mesh patch that is positioned against the peritoneum layer is secured thereagainst by the second holding member while the first holding member is retained against the fascia layer of the abdominal wall.

In accordance with a second preferred methodology using the assembly as disclosed in the second embodiment, the preferred steps comprise inserting the first end of the outer elongated shaft through the mesh layer and through at least the peritoneum layer and the fascia layer of the abdominal wall; deploying the first holding member out the first end of the outer elongated shaft and into a region of the abdominal wall intermediate a skin layer and a fascia layer; pulling the outer elongated shaft out of the abdominal wall; exiting the second holding member out the first end of the outer elongated shaft; applying a tension on the intermediate portion of the first coupler to pull the first holding member and the second holding member towards each other; and locking the locking arrangement about the intermediate portion of the first coupler; whereby a mesh patch that is positioned against the peritoneum layer is secured thereagainst by the second holding member while the first holding member is retained against the fascia layer of the abdominal wall.

In yet another embodiment using the assembly as disclosed in the third embodiment of FIG. 12, the method comprises the steps of inserting the first end of the outer elongated shaft through the mesh layer and through at least the peritoneum layer and the fascia layer of the abdominal wall; deploying the first holding member out the first end of the outer elongated shaft and into a region of the abdominal wall intermediate a skin layer and a fascia layer; pulling the outer elongated shaft out of the abdominal wall; exiting the second holding member out the first end of the outer elongated shaft; inserting the male section into the female section so that the first holding member and the second holding member are in interlocked engagement with each other; whereby a mesh patch that is positioned against the peritoneum layer is secured thereagainst by the second holding member while the first holding member is retained against the fascia layer of the abdominal wall.

It should however also be clear to those skilled in the art that the methodology could be somewhat reversed by the person using the assembly disclosed herein and claimed.

For example, in one alternative embodiment, a method of securing a mesh patch to a surface of a peritoneum layer of an abdominal wall may utilize the assembly disclosed in the first embodiment and comprises the steps of inserting the first end of the outer elongated shaft through the fascia layer of the abdominal wall and thereafter through at least the peritoneum layer and the mesh layer; deploying the first holding member out the first end of the outer elongated shaft; pulling the outer elongated shaft out of the abdominal wall; exiting the second holding member out the first end of the outer elongated shaft; applying tension on the intermediate portion of the coupler to cause the first holding member and the second holding member to be pulled towards each other, and locking the locking arrangement about the intermediate portion of the coupler, whereby a mesh patch that is positioned against the peritoneum layer is secured thereagainst by the first holding member while the second holding member is retained against the fascia layer of the abdominal wall.

In another alternative method of securing a mesh patch to a surface of a peritoneum layer of an abdominal wall, the person or persons performing the method preferably uses an assembly as disclosed in the second embodiment and comprises the steps of inserting the first end of the outer elongated shaft through the fascia layer of the abdominal wall, thereafter through at least the peritoneum layer and through the mesh layer; deploying the first holding member out the first end of the outer elongated shaft; pulling the outer elongated shaft out of the abdominal wall; exiting the second holding member out the first end of the outer elongated shaft; applying a tension on the intermediate portion of the first coupler to pull the first and second holding members towards each other; and locking the locking arrangement about the intermediate portion of the first coupler; whereby a mesh patch that is positioned against the peritoneum layer is secured thereagainst by the first holding member while the second holding member is retained against the fascia layer of the abdominal wall.

In yet a third alternative methodology using the assembly disclosed in the third embodiment, securing a mesh patch to a surface of a peritoneum layer of an abdominal wall may comprise the steps of inserting the first end of the outer elongated shaft through the fascia layer of the abdominal wall, and thereafter at least through the peritoneum layer and the mesh layer; deploying the first holding member out the first end of the outer elongated shaft; pulling the outer elongated shaft out of the abdominal wall; exiting the second holding member out the first end of the outer elongated shaft; inserting the male section into the female section so that the first holding member and the second holding member are in interlocked engagement with each other; whereby a mesh patch that is positioned against the peritoneum layer is secured thereagainst by the first holding member while the second holding member is retained against the fascia layer of the abdominal wall.

It can thus be seen that the present invention provides improved methodologies and assemblies for securing a mesh patch to an abdominal layer and overcomes existing problems in the art. For example, the present invention avoids unnecessary skin incisions. In addition to cosmetic benefits, patients will experience less pain and avoid a potential source for a mesh infection which could otherwise lead to a necessary mesh removal and undesirable patient outcomes. The present invention also provides for more accurate placement of the trans-fascial fixation stitches. Depending on the thickness of the abdominal wall it can be very difficult to tie the knot just above the fascia with the appropriate tension. In the prior art, two (2) passes with a suture passer through the abdominal wall are necessary. However, the present invention allows for precise placement to the fascia with only one pass through the abdominal wall, thus decreasing the chance of injuries to blood vessels and potentially less trauma to the tissue, thus resulting in potentially less pain for the patient.

Furthermore, only basic laparoscopic skills are needed with the present invention. For example, in existing assemblies, the suture needs to be passed through the abdominal wall and removed out of the suture passer. Then the suture passer is pulled back and repassed through the abdominal wall and then the suture is transferred to the suture passer before it can be pulled out, all of which requires advanced laparoscopic skills. However, the present invention eliminates this step and converts this operation from an advanced laparoscopic operation to a basic laparoscopic procedure with less laparoscopic skills needed. Still further, a reduction in operating time is realized by the use of the present invention. That is, currently, in addition to the advanced laparoscopic skills that are necessary for the placement of the trans-fascial stitches, it requires time to place them. This may be multiplied by the use of usually 4, 8 or 12 of these trans-fascial stitches. However, with the present invention, the time needed to complete the procedures contemplated herein are minimized.

In addition, the use of the term “self-tightening” knot is intended to encompass all such knots that functionally carry out the objectives of the invention, and therefore, are intended to include, without limitation, a locking knot, a slip knot and/or any other form of self-tightening knot as known or would be understood in the art.

It is also noted that the present invention has been disclosed in connection with a first coupling position of the mesh layer to the peritoneum layer. In a preferred embodiment, there are preferably at least four (4) such deployments, namely at the 12:00 position, the 3:00 position, the 6:00 position and the 9:00 position of the mesh patch. Therebetween, conventional tack and/or screws may be used to ensure the edges of the mesh patch do not undesirably hang down or about the peritoneum layer. The mesh patch may be inserted through the trocar in any number of known methods, such as rolling it tightly and inserting it therein. And finally, finger holes/supports may be provided at the end of deployment member 30 and on the side of shaft 14 as illustrated in FIGS. 1, 7, 12, (shown exemplary on the right side of each respective figure) although again, because of scale constraints, the actual holes/supports are not illustrated but should be well understood by those skilled in the art.

It is also to be understood that the following claims are intended to cover all of the generic and specific features of the invention described herein and all statements of the scope of the invention which as a matter of language might fall therebetween.

What is claimed is:

1. An assembly for securing a mesh patch to a surface of a peritoneum layer of an abdominal wall, the assembly comprising:
an outer elongated shaft having a first end;
a first holding member at least partially positionable in the outer elongated shaft and a second holding member at least partially positionable in the outer elongated shaft,
a coupler for coupling the first holding member to the second holding member, the coupler comprising a locking arrangement and an intermediate portion;
a deployment member for deploying at least the first holding member out the first end of the outer elongated shaft, wherein the second holding member is also exitable out the first end of the outer elongated shaft;
wherein:
the locking arrangement is lockable about the intermediate portion of the coupler, and
a tension on the intermediate portion of the coupler pulls the first holding member and the second holding member towards each other;
whereby with the locking arrangement locked to the intermediate portion of the coupler, a mesh patch that is positioned against the peritoneum layer is secured thereagainst by one of the first and the second holding members while the other of the first and second holding members is retained against the fascia layer of the abdominal wall.

2. The assembly as claimed in claim 1, comprising means for engaging the locking arrangement while tension is applied to the intermediate portion of the coupler.

3. The assembly as claimed in claim 2, wherein the means for engaging comprises an inner elongated shaft that is positionable in the outer elongated shaft and through which at least a section of the intermediate portion of the coupler extends, wherein the inner elongated shaft facilitates in maintaining the position of the locking arrangement proximate the second holding member as tension is applied to the intermediate portion of the coupler.

4. The assembly as claimed in claim 1, wherein the locking arrangement is a self-tightening knot and wherein the tension on the intermediate portion of the coupler causes the tightening of the self-tightening knot about the intermediate portion of the coupler.

5. The assembly as claimed in claim 2, comprising a tubular section, wherein the outer elongated shaft is positioned within the tubular section, wherein the means for engaging comprises a slot in a front end of the tubular section, wherein the slot maintains the position of the locking arrangement proximate the second holding member as tension is applied to the intermediate portion of the coupler.

6. The assembly as claimed in claim 2, wherein the means for engaging comprises a slot in a front end of the outer elongated shaft, wherein the slot maintains the position of the locking arrangement proximate the second holding member as tension is applied to the intermediate portion of the coupler.

7. The assembly as claimed in claim 3, wherein the inner elongated shaft comprises at least one of:
a rear end out of which the intermediate portion of the coupler is pullable so as to apply tension on the intermediate portion of the coupler so as to cause the first holding member to be pulled towards the second holding member; and
an opening in a side surface out of which the intermediate portion of the coupler is pullable so as to apply tension on the intermediate portion of the coupler so as to cause the first holding member to be pulled towards the second holding member.

8. The assembly as claimed in claim 1, wherein the first holding member is deployed into a region of the abdominal wall intermediate a skin layer and a fascia layer and wherein the mesh patch is secured against the peritoneum layer by the second holding member while the first holding member is retained against the fascia layer of the abdominal wall.

9. An assembly for securing a mesh patch to a surface of a peritoneum layer of an abdominal wall, the assembly comprising:
an outer elongated shaft having a first end;
a first holding member at least partially positionable in the outer elongated shaft and a second holding member at least partially positionable in the outer elongated shaft,
a coupling arrangement for coupling the first holding member to the second holding member, the coupling arrangement comprising:
a first coupler that comprises a first end that is coupled to the first holding member and an intermediate portion that passes through the second holding member; and
a second coupler that comprises an intermediate portion and a locking arrangement that is lockable to the intermediate portion of the first coupler;
a deployment member for deploying at least the first holding member out the first end of the outer elongated shaft, wherein the second holding member is also exitable out the first end of the outer elongated shaft,
wherein:
a tension on the intermediate portion of the first coupler pulls the first and second holding members towards each other; and
locking the locking arrangement on the intermediate portion of the first coupler locks the first holding member in tension relative to the second holding member;
whereby a mesh patch that is positioned against the peritoneum layer is secured thereagainst by one of the first and the second holding members while the other of the first and second holding members is retained against the fascia layer of the abdominal wall.

10. The assembly as claimed in claim 9, comprising means for engaging the locking arrangement while tension is applied to the intermediate portion of the first coupler.

11. The assembly as claimed in claim 10, wherein the means for engaging comprises an inner elongated shaft that is positionable in the outer elongated shaft and through which at least a section of the intermediate portion of the coupler extends, wherein the inner elongated shaft facilitates in maintaining the position of the locking arrangement proximate the second holding member as tension is applied to the intermediate portion of the first coupler.

12. The assembly as claimed in claim 9, wherein:
the intermediate portion of the first coupler passes through the locking arrangement of the second coupler;
the locking arrangement is a self-tightening knot; and
the tension on the intermediate portion of the second coupler causes the tightening of the self-tightening knot about the intermediate portion of the first coupler.

13. The assembly as claimed in claim 10, comprising a tubular section, wherein the outer elongated shaft is positioned within the tubular section, wherein the means for engaging comprises a slot in a front end of the tubular section, wherein the slot maintains the position of the locking arrangement proximate the second holding member as tension is applied to the intermediate portion of the first coupler.

14. The assembly as claimed in claim 11, wherein the inner elongated shaft comprises at least one of:
a rear end out of which the intermediate portion of the coupler is pullable so as to apply tension on the intermediate portion of the first coupler so as to cause the first holding member to be pulled towards the second holding member; and
an opening in a side surface out of which the intermediate portion of the first coupler is pullable so as to apply tension on the intermediate portion of the first coupler so as to cause the first holding member to be pulled towards the second holding member.

15. The assembly as claimed in claim 9, wherein the first holding member is deployed into a region of the abdominal wall intermediate a skin layer and a fascia layer and wherein the mesh patch is secured against the peritoneum layer by the second holding member while the first holding member is retained against the fascia layer of the abdominal wall.

16. An assembly for securing a mesh patch to a surface of a peritoneum layer of an abdominal wall, the assembly comprising:
an outer elongated shaft having a first end;
a first holding member at least partially positionable in the outer elongated shaft and a second holding member at least partially positionable in the outer elongated shaft,
a coupling arrangement for coupling the first holding member to the second holding member, the coupling arrangement comprising:
a female section coupled to one of the first and second holding members, and a male section coupled to the other of the first and second holding members, wherein the male and female sections have complementary locking structures for interlocking engagement therebetween; and
a coupler that includes a first end that is coupled to the first holding member and an intermediate portion that passes through the female section, male section and the second holding member;
a deployment member for deploying at least the first holding member out the first end of the outer elongated shaft, wherein the second holding member is also exitable out the first end of the outer elongated shaft,
wherein the insertion of the male section into the female section causes the first holding member and the second holding member to be interlocked with each other;
whereby a mesh patch that is positioned against the peritoneum layer is secured thereagainst by one of the first and the second holding members while the other of the first and second holding members is retained against the fascia layer of the abdominal wall.

17. The assembly as claimed in claim 16, comprising means for urging the interlocking engagement of the female and male sections, wherein the means for urging comprises an inner elongated shaft that is positionable in the outer elongated shaft and through which at least a section of the intermediate portion of the coupler extends.

18. The assembly as claimed in claim 17, wherein the inner elongated shaft comprises at least one of:
a rear end out of which the intermediate portion of the coupler is pullable so as to apply tension on the intermediate portion of the coupler so as to cause the first holding member to be pulled towards the second holding member; and
an opening in a side surface out of which the intermediate portion of the coupler is pullable so as to apply tension on the intermediate portion of the coupler so as to cause the first holding member to be pulled towards the second holding member.

19. The assembly as claimed in claim 16, comprising means for urging the interlocking engagement of the female and male sections, wherein the means for urging comprises a front end of the outer elongated shaft.

20. The assembly as claimed in claim 16, comprising a tubular section, wherein the outer elongated shaft is positioned within the tubular section, further comprising means for urging the interlocking engagement of the female and male sections, wherein the means for urging comprises a front end of the tubular section.

21. The assembly as claimed in claim 16, wherein the coupling arrangement comprises a locking coupler that comprises an intermediate portion and a locking arrangement that is lockable to the intermediate portion of the coupler.

22. The assembly as claimed in claim 21, wherein:
the intermediate portion of the coupler passes through the locking arrangement of the locking coupler;
the locking arrangement is a self-tightening knot; and
a tension on the intermediate portion of the locking coupler causes the tightening of the self-tightening knot about the intermediate portion of the coupler.

23. The assembly as claimed in claim 16, wherein:
the female section is coupled to the first holding member and is rotatable relative thereto, and wherein the female section is in a collapsed position when positioned in the outer elongated shaft;
the male section is coupled to the second holding member and is rotatable relative thereto, and wherein the male section is in a collapsed position when positioned in the outer elongated shaft; and
wherein when the first holding member is in the region of the abdominal wall intermediate the skin layer and the fascia layer, the female section rotates to an extended position and wherein when the second holding member is deployed out the first end of the outer elongated shaft the male section rotates to an extended position.

24. The assembly as claimed in claim 23, wherein:
the female section is hingedly coupled to the first holding member and the male section is hingedly coupled to the second holding member; and
wherein the female section is lockable in its extended position and the male section is lockable in its extended position.

25. The assembly as claimed in claim 16, wherein the mesh patch is secured against the peritoneum layer by the second holding member while the first holding member is retained against the fascia layer of the abdominal wall.

26. A method of securing a mesh patch to a surface of a peritoneum layer of an abdominal wall utilizing an assembly that comprises an outer elongated shaft having a first end; a first holding member at least partially positionable in the outer elongated shaft and a second holding member at least partially positionable in the outer elongated shaft; a coupler for coupling the first holding member to the second holding member, the coupler having a locking arrangement and an intermediate portion; a deployment member for deploying at least the first holding member out the first end of the outer elongated shaft and into a region of the abdominal wall intermediate a skin layer and a fascia layer, wherein the second holding member is also exitable out the first end of the outer elongated shaft; wherein the locking arrangement is lockable about the intermediate portion of the coupler, and a tension on the intermediate portion of the coupler pulls the first holding member and the second holding member towards each other; wherein the method comprises the steps of:
inserting the first end of the outer elongated shaft through the mesh layer and through at least the peritoneum layer and the fascia layer of the abdominal wall;
deploying the first holding member out the first end of the outer elongated shaft and into a region of the abdominal wall intermediate a skin layer and a fascia layer;
pulling the outer elongated shaft out of the abdominal wall;
exiting the second holding member out the first end of the outer elongated shaft;
applying tension on the intermediate portion of the coupler to cause the first holding member and the second holding member to be pulled towards each other, and
locking the locking arrangement about the intermediate portion of the coupler,
whereby a mesh patch that is positioned against the peritoneum layer is secured thereagainst by the second holding member while the first holding member is retained against the fascia layer of the abdominal wall.

27. The method as claimed in claim 26, including the step of maintaining the position of the locking arrangement proximate the second holding member as tension is applied to the intermediate portion of the coupler.

28. The method as claimed in claim 26, wherein the locking arrangement is a self-tightening knot, including the step of:
applying the tension on the intermediate portion of the coupler to tighten the self-tightening knot about the intermediate portion of the coupler.

29. A method of securing a mesh patch to a surface of a peritoneum layer of an abdominal wall utilizing an assembly comprising an outer elongated shaft having a first end; a first holding member at least partially positionable in the outer elongated shaft and a second holding member at least partially positionable in the outer elongated shaft; a coupling arrangement for coupling the first holding member to the second holding member, the coupling arrangement comprising a first coupler that comprises a first end that is coupled to the first holding member and an intermediate portion that passes through the second holding member, and a second coupler that comprises an intermediate portion and a locking arrangement that is lockable to the intermediate portion of the first coupler; a deployment member for deploying at least the first holding member out the first end of the outer elongated shaft and into a region of the abdominal wall intermediate a skin layer and a fascia layer, wherein the second holding member is also exitable out the first end of the outer elongated shaft; wherein a tension on the intermediate portion of the coupler pulls the first holding member and the second holding member towards each other; and locking the locking arrangement on the intermediate portion of the first coupler locks the first holding member in tension relative to the second holding member; wherein the method comprises the steps of:
inserting the first end of the outer elongated shaft through the mesh layer and through at least the peritoneum layer and the fascia layer of the abdominal wall;
deploying the first holding member out the first end of the outer elongated shaft and into a region of the abdominal wall intermediate a skin layer and a fascia layer;
pulling the outer elongated shaft out of the abdominal wall;
exiting the second holding member out the first end of the outer elongated shaft;
applying a tension on the intermediate portion of the first coupler to cause the first holding member and the second holding member to be pulled towards each other; and
locking the locking arrangement about the intermediate portion of the first coupler;
whereby a mesh patch that is positioned against the peritoneum layer is secured thereagainst by the second holding member while the first holding member is retained against the fascia layer of the abdominal wall.

30. The method as claimed in claim 29, wherein the locking arrangement is a self-tightening knot and wherein the intermediate portion of the first coupler passes through the self-tightening knot of the second coupler, including the step of:
applying a tension on the intermediate portion of the second coupler to cause the locking of the self-tightening knot about the intermediate portion of the first coupler.

31. A method for securing a mesh patch to a surface of a peritoneum layer of an abdominal wall using an assembly comprising an outer elongated shaft having a first end; a first holding member at least partially positionable in the outer elongated shaft and a second holding member at least partially positionable in the outer elongated shaft; a coupling arrangement for coupling the first holding member to the second holding member, the coupling arrangement comprising a female section coupled to one of the first and second holding members, and a male section coupled to the other of the first and second holding members, wherein the female section and the male section have complementary locking structures for interlocking engagement therebetween; and a coupler that includes a first end that is coupled to the first holding member and an intermediate portion that passes through the female section, the male section and the second holding member; a deployment member for deploying at least the first holding member out the first end of the outer elongated shaft and into a region of the abdominal wall intermediate a skin layer and a fascia layer, wherein the second holding member is also exitable out the first end of the outer elongated shaft; wherein the method comprises the steps of:
inserting the first end of the outer elongated shaft through the mesh layer and through at least the peritoneum layer and the fascia layer of the abdominal wall;
deploying the first holding member out the first end of the outer elongated shaft and into a region of the abdominal wall intermediate a skin layer and a fascia layer;
pulling the outer elongated shaft out of the abdominal wall;
exiting the second holding member out the first end of the outer elongated shaft;
inserting the male section into the female section so that the first holding member and the second holding member are in interlocked engagement with each other;
whereby a mesh patch that is positioned against the peritoneum layer is secured thereagainst by the second holding member while the first holding member is retained against the fascia layer of the abdominal wall.

32. The method as claimed in claim 31, including the step of:
applying a tension on the intermediate portion of the coupler to pull the first and second holding members towards each other as the male section is urged into the female section.

33. The method as claimed in claim 31, comprising a second coupler that comprises an intermediate portion and a locking arrangement that is lockable to the intermediate portion of the first coupler; including the step of:
locking the locking arrangement about the intermediate portion of the first coupler after inserting the male section into the female section.

34. The method as claimed in claim 32, wherein the locking arrangement is a self-tightening knot and wherein the intermediate portion of the first coupler passes through the self-tightening knot of the second coupler, including the step of:
applying a tension on the intermediate portion of the second coupler to cause the locking of the self-tightening knot about the intermediate portion of the first coupler.
